(19) United States
(12) Patent Application Publication
(10) Pub. No.: US 2021/0030467 A1
(43) Pub. Date: Feb. 4, 2021

(54) DEVICES AND METHODS FOR DELIVERING MATERIAL INTO A BIOLOGICAL TISSUE OR CELL

(71) Applicant: Flagship Pioneering Innovations V, Inc., Cambridge, MA (US)

(72) Inventors: Michael Mee, Boston, MA (US); Adam Rago, Somerville, MA (US); Geoffrey von Maltzahn, Somerville, MA (US); John Miles Milwid, Winchester, MA (US); Jacob Rosenblum Rubens, Cambridge, MA (US); Michael J. Cima, Winchester, MA (US)

(73) Assignee: Flagship Pioneering Innovations V, Inc., Cambridge, MA (US)

(21) Appl. No.: 16/968,270

(22) PCT Filed: Feb. 8, 2019
(86) PCT No.: PCT/US2019/017268

§ 371 Date: Aug. 7, 2020

Related U.S. Application Data
(60) Provisional application No. 62/629,322, filed on Feb. 12, 2018.

(51) Int. Cl. A61B 18/14; A61B 90/00; A61B 90/30

(52) U.S. Cl. CPC A61B 18/1492; A61B 2018/0022; A61B 90/30; A61B 90/361

(57) ABSTRACT

One aspect of the invention provides a device for delivering material into a biological tissue. The device includes: a reservoir for the material; and a material delivery unit in connection with the reservoir configured to transfer the material from the reservoir to the tissue. Another aspect of the invention provides an implantable or insertable delivery device for delivery of material across or into a biological tissue in a subject. The device includes: a reservoir for holding the material; and a tissue-penetrating member.

### CROSS-REFERENCE TO RELATED APPLICATIONS

This application claims the benefit of priority to U.S. Provisional Patent Application Ser. No. 62/629,322, filed Feb. 12, 2018. The entire content of this application is hereby incorporated by reference herein.

### BACKGROUND OF THE INVENTION

Cells include a variety of subcellular components, also known as organelles, that have a specific function.

### SUMMARY OF THE INVENTION

One aspect of the invention provides a device for delivering material into a biological tissue. The device includes: a reservoir for the material; and a material delivery unit in connection with the reservoir configured to transfer the material from the reservoir to the tissue.

This aspect of the invention can have a variety of embodiments. The reservoir have a volumetric capacity in the range of about 0.5 mL to about 500 mL. The reservoir can be in connection with a metering unit. The reservoir can be in connection with a pump. The reservoir can be configured to maintain a specific temperature, pressure, or viscosity of the material prior to delivery.

The reservoir can be configured to allow preparation of the material prior to delivery. The preparation can include one or more selected from the group consisting of: mixing, temperature, and viscosity optimization for delivery.

The reservoir can be pressurized to aerosolize the material.

The reservoir can include a permeable membrane. The permeable membrane can include one or more selected from the group consisting of: a natural polymer, a synthetic polymer, a stent with a polymer coating, and a hydrogel/polymer matrix. The permeable membrane can have a pore size of less than about 10 μm, less than about 9 μm, less than about 8 μm, less than about 7 μm, less than about 6 μm, less than about 5 μm, less than about 4 μm, less than about 3 μm, less than about 2 μm, less than about 1 μm, less than about 500 nm, less than about 200 nm, less than about 100 nm, and less than about 50 nm.

The material delivery unit can include electrical circuitry configured to generate at least one selected from the group consisting of: a thermal change, a physical contact force, an ultrasonic frequency, an osmotic change, a pressure change, a photothermal pulse, a magnetic field, an electromagnetic field, an electric field, and an electrical pulse through the reservoir.

The material delivery unit can include a material-dispensing member that transfers the material from the reservoir to the tissue. The material-dispensing member can be selected from the group consisting of: a metering unit and a pump.

The material delivery unit can be configured for insertion into a patient's body.

The device can be implantable or insertable into a subject.

The material delivery unit can include a tissue-penetrating member for piercing tissue. The tissue-penetrating member can include a single injector. The tissue-penetrating member can be configured to pierce the tissue to a preselected depth. The preselected depth can be suitable for one or more selected from the group consisting of: transdermal, transendothelial, transepithelial, atherosclerolic, extravesicular, arteriole, venous, and peritoneal applications. The tissue-penetrating member can include an array of injectors. The array of injectors can be configured to pierce the tissue at a uniform depth or multiple depths.

The material delivery unit can include one or more selected from the group consisting of: a stent, tubing, a balloon, and a microneedle.

The material delivery unit can include a catheter fluidly connected to the reservoir. The catheter can be configured to be removably connected to the reservoir. The catheter can be a Peripherally Inserted Central Catheter (PICC). The catheter can further include a balloon.

The device can further include a plunger configured to expel the material out of the reservoir into the material delivery unit.

The device can further include a tissue-conditioning apparatus. The material delivery unit can be configured to deliver the material into the tissue upon application of the tissue-conditioning apparatus. The tissue-conditioning apparatus can be adapted and configured to alter the tissue to increase uptake of the material within the tissue. The tissue-conditioning apparatus can include a light source. The tissue-conditioning apparatus can be configured to abrade, puncture, or thermally ablate a surface of the tissue. The tissue-conditioning apparatus can be configured to expose the tissue to at least laser or high-frequency radio waves. The tissue-conditioning apparatus can be configured to expose the tissue to at least one selected from the group consisting of: a thermal change, a physical contact force, a shear contact force, an ultrasonic frequency, a photothermal pulse, a magnetic field, an electromagnetic field, an electric field, and an electrical pulse.

The device can further include an imaging device. The imaging device can be selected from the group consisting of: a camera, an X-ray imaging detector, ultrasound, a computed tomography (CT) device, a magnetic resonance imaging (MRI) device, an arthroscopic device, and an endoscope.

The device can have a largest cross-sectional profile selected from the group consisting of: less than about 10 mm², less than about 5 mm², less than about 4 mm², less than about 3 mm², less than about 2 mm², or less than about 1 mm².

The reservoir can be hermetically sealed and the material delivery unit is configured to require activation to release the material from the reservoir. The activation can be an electric pulse.

The device can further include a tissue stabilizer including a tissue contacting member. The tissue stabilizer can be operatively associated with the material delivery unit. The tissue stabilizer can be adapted and configured to hold the tissue during actuation of the material delivery unit.

The device can further include a closed-loop system. The closed-loop system can be an apheresis device.

The device can further include a sensor configured to obtain a measurement of the tissue.

The device can further include a computer. The computer can be programmed to perform one or more functions selected from the group consisting of: storing information, regulating delivery, adjusting delivery in response to a measurement, and adjusting delivery in response to a measurement from a sensor.

The device can be configured for delivery to a specific tissue type. The specific tissue type can be selected from the group consisting of: muscle, epithelial tissue, connective tissue, and nervous tissue.

The device can be configured for delivery to a specific body location. The specific body location can be selected from the group consisting of: cardiovasculature, circulatory system, digestive tract, excretory organs, CNS, lymph nodes, immune organs, musculoskeletal tissues, respiratory organs, reproductive organs, and a placenta.

Another aspect of the invention provides an implantable or insertable delivery device for delivery of material across or into a biological tissue in a subject. The device includes: a reservoir for holding the material; and a tissue-penetrating member.

This aspect of the invention have a variety of embodiments. The reservoir can be in connection with a metering unit. The reservoir can be configured to maintain a specific temperature, pressure, or viscosity of the material prior to delivery. The reservoir can be configured to perform one or more steps to the material prior to delivery. The one or more steps can be selected from the group consisting of: preparation, mixing, temperature optimization for delivery, and viscosity optimization for delivery.

The device can further include a plunger configured to expel the material out of the reservoir into the tissue.

The tissue-penetrating member can be adapted and configured to pierce tissue. The tissue-penetrating member can include a single injector. The tissue-penetrating member can be configured to pierce the tissue to a preselected depth.

The tissue-penetrating member can include an array of injectors. The array of injectors can pierce the tissue at a uniform depth or multiple depths.

The tissue-penetrating member can be a catheter. The catheter can be configured to be removably connected to the reservoir. The catheter can be a Peripherally Inserted Central Catheter (PICC).

Another aspect of the invention provides a system for delivering material into a biological tissue. The system includes: a tissue conditioning apparatus; a reservoir for the material; and a material delivery unit in connection with a reservoir configured to transfer the material from the reservoir to the tissue.

This aspect of the invention have a variety of embodiments. The biological tissue can be skin.

The reservoir can have a volumetric capacity in the range of about 0.5 mL to about 500 mL. The reservoir can be in connection with a metering unit. The reservoir can be in connection with a pump. The reservoir can be configured to maintain a specific temperature, pressure, or viscosity of the material prior to delivery. The reservoir can be configured to perform one or more steps to the material prior to delivery, the one or more steps selected from the group consisting of: preparation, mixing, temperature optimization for delivery, and viscosity optimization for delivery.

The tissue conditioning apparatus can include a light source. The tissue conditioning apparatus can be configured to abrade, puncture, or thermally ablate a surface of the tissue. The tissue conditioning apparatus can be configured to expose the tissue to at least one selected from the group consisting of: an electric field, a magnetic field, an electromagnetic field, a photothermal energy, and an ultrasonic frequency.

The reservoir can include a membrane. The material delivery unit can aid absorption of the material into the tissue. The membrane can be selected from the group consisting of: a transdermal patch and a sublingual patch.

Another aspect of the invention provides a delivery device for material transfer across a membrane-enclosed object comprising a reservoir and a microfluidic channel. Movement of the membrane-enclosed object through the microfluidic channel permeabilizes the membrane to allow movement of a material through the membrane This aspect of the invention can have a variety of embodiments. The membrane-enclosed object can have a maximal cross-sectional dimension selected from the group consisting of: less than 5 less than 4 and less than 3 The membrane-enclosed object can be selected from the group consisting of: a cell, a microparticle, a vesicle, an organelle, and an endosome.

The microfluidic channel can contact the membrane to permeabilize the membrane. The microfluidic channel can have a diameter of at least 10% of the maximum cross-sectional dimension of a cell.

The membrane-enclosed object can be selected from the group consisting of: a cell, a microparticle, a vesicle, an organelle, and an endosome.

The device can further include electrical circuitry. The electrical circuitry can be configured to generate an electrical pulse through the microfluidic channel. The electrical circuitry can be configured to generate at least one selected from the group consisting of: a thermal change, a physical contact force, an ultrasonic frequency, an osmotic change, a pressure change, a photothermal pulse, a magnetic field, an electromagnetic field, an electric field, and an electrical pulse through the microfluidic channel. The electrical circuitry can be configured to allow transfer of the material into the object at a specific ratio of material-to-object as measured by quantity, by mass, or by volume. The ratio of material-to-object can be in a range of about 1:1 to about 20:1. The electrical circuitry can be configured to maintain a specific temperature, pressure, or viscosity of the material prior to movement through the membrane.

The device can further include a pump configured to maintain a flow through the microfluidic channel.

The reservoir can include an inlet and an outlet for fluidic movement of cells into and out of the reservoir.

Another aspect of the invention provides a system for material transfer into a plurality of membrane-enclosed objects comprising a reservoir and a microfluidic channel. The microfluidic channel contacts a membrane of the membrane-enclosed objects to permeabilize the membrane and allow movement of a material through the membrane.

This aspect of the invention can have a variety of embodiments. The membrane-enclosed object can be selected from the group consisting of: a cell, a microparticle, a vesicle, an organelle, and an endosome.

The microfluidic channel can have a diameter of at least 10% of the maximum cross-sectional dimension of a cell to permeabilize the membrane. The microfluidic channel can be capable of permeabilizing at least 100 cells per minute, 1,000 cells per minute, 10,000 cells per minute, or 100,000 cells per minute.

The system can be configured to facilitate transfer of the material into the membrane-enclosed objects at a specific ratio of material-to-object as measured by quantity, by mass, or by volume. The ratio of material-to-object can be in a range of about 1:1 to about 20:1.

The system can be configured to maintain a specific temperature, pressure, viscosity of the material prior to movement through the membrane.

Another aspect of the invention provides a system for material transfer into a plurality of membrane-enclosed objects comprising a reservoir and electrical circuitry configured to generates at least one selected from the group consisting of: an electric field, a magnetic field, an electromagnetic field, a photothermal pulse, and an ultrasonic frequency in the reservoir to permeabilize a membrane of the object and allow movement of a material through the membrane.

This aspect of the invention can have a variety of embodiments. The electrical circuitry can be configured to facilitate transfer of the material into the membrane-enclosed objects at a specific ratio of material-to-object as measured by quantity, by mass, or by volume. The ratio of material-to-object can be in a range of about 1:1 to about 20:1.

The electrical circuitry can be configured to generate a temperature, pressure, or viscosity change in the reservoir to facilitate movement of the material through the membrane.

Another aspect of the invention provides a delivery device comprising a reservoir and a membrane-penetrating apparatus. The membrane-penetrating apparatus is configured to induce movement of a material through a membrane of a membrane-enclosed object in the reservoir.

This aspect of the invention can have a variety of embodiments. The reservoir can include an inlet and an outlet for fluidic movement of the object into and out of the reservoir.

The device can be configured to maintain a specific temperature, pressure, viscosity of the material prior to movement through the membrane.

The device can further include a pump configured to maintain a flow.

The penetrating apparatus can be an injector. The injector can be configured to pierce the membrane-enclosed object to: inject material into the membrane-enclosed object, extract material from the membrane-enclosed object, or inject material into the membrane-enclosed object and extract material from the membrane-enclosed object.

The device can be a high-throughput injector. The high-throughput injector can be capable of injecting at least 100 objects per minute, 1,000 objects per minute, 10,000 objects per minute, or 100,000 objects per minute.

The device can further include a system configured to collect and exchange biological fluid. The system can be an apheresis device. The biological fluid can be selected from the group consisting of: blood and bodily fluid.

The device can further include a detection device configured to monitor the movement of the material and obtain cellular image data. The detection device can further include an imaging device. The imaging device can be a camera.

The device can further include a computer. The computer can be programmed to perform one or more functions selected from the group consisting of: for storing information and regulating delivery. The computer can be an automated machine configured to follow machine-readable instructions that facilitate the transport of the objects, injection into the objects and extraction from the objects.

Another aspect of the invention provides a system for automated extracorporeal injection comprising: (a) a collection system; (b) a computer including control software for motion control and image processing; (c) a control device to control motion and immobilize one or more membrane-enclosed objects in a desired position; and (d) an injection mechanism. The control device and the injection mechanism are linked to the computer to facilitate the injection of material into the objects.

This aspect of the invention can have a variety of embodiments. The system can further include a microscope for viewing position of the injection mechanism relative to the objects.

The collection system can be an apheresis device.

Another aspect of the invention provides a high-throughput system for automated injection comprising: (a) a computer including control software for motion control and image processing; (b) a control device to control motion and immobilize one or more membrane-enclosed objects in a desired position; (c) an injection mechanism; and (d) a microscope for viewing the position of the injection mechanism relative to the objects. The control device, the injection mechanism and the microscope are linked to the computer to enable the injection into the objects.

Another aspect of the invention provides a delivery device for delivering cells, subcellular components, fusogens, fusosomes, or fusosome compositions into a biological tissue. The delivery device includes: a reservoir comprising one or more selected contents from the group consisting of: cells, subcellular components, fusogens, fusosomes, and fusosome compositions; and a material delivery unit in connection with the reservoir. The material delivery unit is configured to transfer the contents from the reservoir to the tissue.

Another aspect of the invention provides a method of delivering cells, subcellular components, fusogens, fusosomes, or fusosome compositions into a biological tissue. The method includes: positioning a delivery device as described herein adjacent to, within, or partially within biological tissue; and controlling the delivery device to transfer the cells, subcellular components, fusogens, fusosomes, or fusosome compositions from the reservoir to the biological tissue.

Another aspect of the invention provides a delivery device for transfer of subcellular components, fusogens, fusosomes, or fusosome compositions across one or more membrane-enclosed objects. The delivery device includes: a first reservoir adapted and configured to hold unpermeabilized membrane-enclosed objects; a permeabilizing module in communication with the first reservoir; and a second reservoir containing subcellular components, fusogens, fusosomes, or fusosome compositions. The second reservoir is in communication with the first reservoir.

Another aspect of the invention provides a method of delivering subcellular components, fusogens, fusosomes, or fusosome compositions into one or more membrane-enclosed objects. The method includes: introducing one or more membrane-enclosed objects into the first reservoir of a delivery device as described herein; and controlling the permeabilizing module to permeabilize the membrane of the one or more membrane-enclosed objects to allow movement of the subcellular components, fusogens, fusosomes, or fusosome compositions through the membrane; and contacting the one or more membrane-enclosed objects with the subcellular components, fusogens, fusosomes, or fusosome compositions from the second reservoir.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a delivery device according to an embodiment of the invention.

FIGS. 2A and 2B depict passive delivery devices according to embodiments of the invention.

FIG. 3 depicts the use of the Ommaya reservoir for material delivery according to an embodiment of the invention.

FIG. 4 depicts a delivery device according to an embodiment of the invention.

FIGS. 5 and 6 depict delivery devices according to embodiments of the invention.

### DETAILED DESCRIPTION OF THE INVENTION

Embodiments of the invention provide a variety of devices and methods for administration of various materials into a biological tissue.

### Administration of Subcellular Components

Embodiments of the invention are particularly useful for the administration of subcellular components such as mitochondria. Compositions including isolated subcellular components such as mitochondria are described in U.S. Patent Application Publication No. 2017/0151287.

Embodiments of the invention can be utilized, in whole or in part, to deliver chondrisomes, chondrisome preparations, fusogens, fusosomes, and/or fusosome compositions, as further described in the Appendix.

### Active Delivery Devices

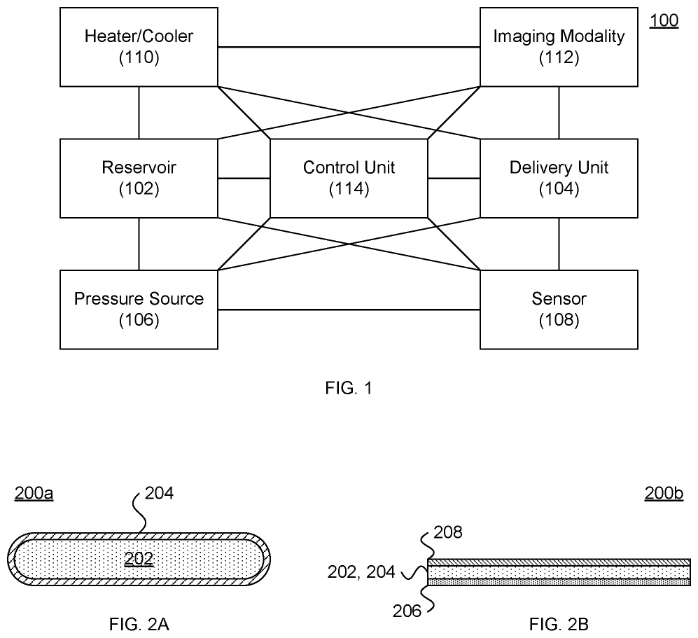

Referring now to FIG. 1, one embodiment of the invention provides a delivery device 100 including a reservoir 102 and a delivery unit 104. The delivery unit 104 can be in communication with (e.g., fluid communication through coupling to) the reservoir such that the material to be delivered passes from the reservoir 102 through the delivery unit 104 and exits into or proximate to the desired location.

The reservoir 102 can include any vessel capable of holding a fluid. In some embodiments, the reservoir is closed to the atmosphere, except for through the delivery unit. Exemplary reservoirs 102 include syringes, tanks, pouches, bladders, and the like. In one embodiment, the reservoir 102 has a volumetric capacity between about 0.5 mL and about 500 mL, and any value in between.

Delivery unit 104 can include any vessel capable of conveying a fluid. For example, delivery unit 104 can include one or more needles (e.g., having sizes between about 7 gauge and about 34 gauge, and any value in between), cannulae, catheters, microneedles, and the like adapted to pierce and/or pass through a tissue surface.

In some embodiments, the delivery device 100 is a double-barreled syringe such disclosed in U.S. Pat. No. 8,074,843 and U.S. Patent Application Publication No. 2015/0112248. Such double-barreled devices enable simultaneous and/or sequential injection of multiple substances and/or withdrawal of fluids from tissue.

In some embodiments, the delivery unit 104 includes or is coupled with or in proximity to one or more retaining members that can engage tissue prior to and/or during administration of a fluid to the tissue. For example, a needle or cannula can be introduced through a sheath of the tissue stabilizer disclosed on U.S. Patent Application Serial No. 2004/0082837 after tissue contacting members engage the target tissue.

In some embodiments, the reservoir 102 and the delivery unit 104 are incorporated within an autoinjector configured to pierce a tissue and/or expel a substance with limited actions by a user. Various autoinjectors are described in U.S. Pat. No. 8,747,357.

In some embodiments, the reservoir 102 and/or the delivery unit 104 are or are incorporated within an implantable device.

Delivery device 100 can further include a pressure source 106. Exemplary pressure sources 106 include plungers such as used in syringes, springs, pumps, mechanical actuators, electrical actuators, electromechanical actuators (e.g., motors, servomotors), pressurized tanks or cartridges, and the like. In some embodiments, the pressure source 106 acts directly on the reservoir 102 (e.g., by compressing or increasing pressure within the reservoir 102). In order embodiments, the pressure source 106 acts indirectly on the reservoir 102 (e.g., by inducing flow in the delivery unit to draw a fluid out of the reservoir 102 (e.g., through the Venturi effect or actuation of a pump positioned along delivery unit 104).

Delivery device 100 can further include one or more sensors 108 that can be configured to assess a condition of a subject and/or the delivery device 100. For example, the sensor 108 can include a temperature sensor configured to measure a temperature of the subject and/or the delivery device 100. The sensor 108 can provide feedback regarding the positioning of delivery device 100. For example, a location of a plunger (e.g., as measured through an optical sensor and/or control of a servomotor) can be utilized to deliver a desired amount of a substance.

Delivery device 100 can further include one or more heaters and/or coolers 110 that can be configured to maintain a desired temperature, pressure, and/or viscosity of the substance within the reservoir 102 (which can be measured by sensor 108). Exemplary heaters/coolers 110 include cooling devices include thermoelectric devices (e.g., Peltier or Ohmic devices), adiabatic cooling devices, fluid-cooled units that communicate with an external heat exchanger, and cryogenic devices that utilize cooled gases such as nitrogen or carbon dioxide to produce the desired low temperatures.

Delivery device 100 can also include one or more imaging modalities 112 adapted and configured to facilitate placement of delivery unit 104 in a desired location. The imaging modality can be an active or passive device. Examples of passive devices include radiopaque markers that can be visualized using one or more other imaging modalities such as ultrasound, X-ray, and the like. Examples of active imaging modalities include ultrasound transducers, cameras (e.g., fiber optics traveling from delivery unit to an external component, charge-coupled devices located on or adjacent to deliver unit, and the like), light sources, laser sources, and the like.

Delivery device 100 can also include one or more controller 114. The control unit 114 can be integrated within the same unit as other components 102, 104, 106, 108, 110, 112, e.g., in an implantable device. In another embodiment, one or more controllers 114 can be external to other components 102, 104, 106, 108, 110, 112 (and sometimes an additional controller 114) and communicate with the other components 102, 104, 106, 108, 110, 112, 114 via one or more wired or wireless communication technologies.

Controller 114 can include a processor device (or central processing unit “CPU”), a memory device, a storage device, a user interface, a system bus, and/or a communication interface.

The controller 114 can, thus, provide for executing processes, by itself and/or in cooperation with one or more additional devices, that can include algorithms for controlling various components of the light sources and photodetector(s) in accordance with the present invention. Controller 108 can be programmed or instructed to perform these processes according to any communication protocol and/or programming language on any platform. Thus, the processes can be embodied in data as well as instructions stored in a memory device and/or storage device or received at a user interface and/or communication interface for execution on a processor.

The controller 108 can control the operation of the system components in a variety of ways. For example, controller 108 can modulate the level of electricity provided to a component. Alternatively, the controller 108 can transmit instructions and/or parameters a system component for implementation by the system component.

### Passive Delivery Devices

Referring now to FIGS. 2A and 2B, other embodiments of the invention provides passive delivery devices 200a, 200b in which a therapeutic 202 is provided within a storage medium 204.

Various storage media 204 can permit passive release of the therapeutic 202 at various rates.

In one embodiment, the storage medium 204 is a permeable membrane configured to allow crossing by the therapeutic 202 (e.g., mitochondria). In one embodiment, the permeable membrane is a porous membrane. Porosity can be measured in effective terms, i.e., the size of particles that will cross the membrane, and/or in absolute terms, i.e., the measured dimension of the pores. Exemplary pore sizes range between about 50 nm and about 10 μm, and any value in between. In other embodiments, the therapeutic 202 can diffuse across the permeable membrane.

In some embodiments such as depicted in FIG. 2B, the storage medium 204 is a polymer that can release the therapeutic 208 over time. Suitable polymers include poloxamers such as poloxamer 188. The polymer and therapeutic can be fabricated as a transdermal patch (e.g., with an adhesive 207 as described in U.S. Patent Application Publication No. 2016/0045158), a subdermal implant, a suppository, and the like. The transdermal patch can include an impermeable cover 208 (e.g., a foil layer).

In some embodiments, the storage medium is a hydrogel/polymer matrix. Exemplary polymers include natural and synthetic polymers such as: polyglycolide (PGA), poly(L-lactic acid) (PLLA), poly-L/D-lactide (PLDLA), poly(l-lactide-co-glycolide) (PLGA), PLGA-collagen matrices, polydioxanone (PDO or PDS), poly(ε-caprolactone) (PCL), poly(DL-lactide) (PDLLA), poly(D,L-lactide-co-ε-caprolactone) (PDLLA-CL), poly(glycolide-co-ε-caprolactone) (PGCL), poly(L-lactide-co-caprolactone) (PLCL), poly(ethylene glycol) (PEG), poly(caprolactone-co-trimethylene carbonate) (PCLTMC), poly(3-hydroxybutyrate)3-hydroxyvalerate (PHBHV), poly(ester urethane) (PEU), polyurethane (PU), lysine diisocyanate (LDI)-based polyurethane (PU), poly(ortho ester) (POE), polyanhydrides, polycyanoacrylate (PCA), collagen, hyaluronic acid (HA), viscous hyaluronic acid (HA), high molecular weight viscous hyaluronic acid (HA), polysulfone (PS), polypropylene (PP), polyvinyl alcohol (PVA), polylactide (PLA), poly(propylene fumarate) (PPF), polyhydroxyalkanoates (PHA), poly(ether ester) (PEE), poly(ethylene oxide) (PEO), polybutylene terephthalate (PBT), poly(acrylic acid) (PAA), polyacrylamide (PAam), polymethylmethacrylate (PMMA), poly(trimethylene carbonate) (PTMC), polydimethylsiloxane (PDMS), polytetrafluoroethylene (PTFE), poly(ethylene-co-vinylacetate) (PEVA), poly(lactic acid-glycolic acid) (PLAGA), poly(N-isopropylacrylamide) (PNIPAAm), poly(dimethylaminoethylmethacrylate hydrochloride) (PDMAEM), poly(l-lactide-co-ε-caprolactone) (PLLA-CL), and the like. Other exemplary polymers are described in Brahatheeswaran Dhandayuthapani et al., “Polymeric Scaffolds in Tissue Engineering Application: A Review”, International Journal of Polymer Science 290602 (2011).

Other exemplary hydrogels are described in Ibrahim M. El-Sherbiny & Magdi H. Yacoub, “Hydrogel scaffolds for tissue engineering: Progress and challenges”, 2013(3) Glob. Cardiol. Sci. Pract. 316-42 (2013).

Other exemplary scaffold materials are described in A. Hasan, “Engineered biomaterials to enhance stem cell-based cardiac tissue engineering and therapy”, 16(7) Macromol. Biosci. 958-77 (July 2016); C. Soler-Botija et al., “A bird's-eye view of cell therapy and tissue engineering for cardiac regeneration”, 1254 Ann. N.Y. Acad. Sci. 57-65 (April 2012); Hui Yun Zhou et al., “Glycerophosphate-based chitosan thermosensitive hydrogels and their biomedical applications”, 117 Carbohydrate Polymers 524-36 (Mar. 6, 2015); C. Vinatier et al., “Cartilage and bone tissue engineering using hydrogels”, 16(4 Suppl.) Biomed. Mater. Eng. S107-13 (2006); and U. Bhardwaj et al., “A review of the development of a vehicle for localized and controlled drug delivery for implantable biosensors”, 2(6) J. Diabetes Sci. Technol. 1016-29 (November 2008).

### Integrated Piercing and Delivery Devices

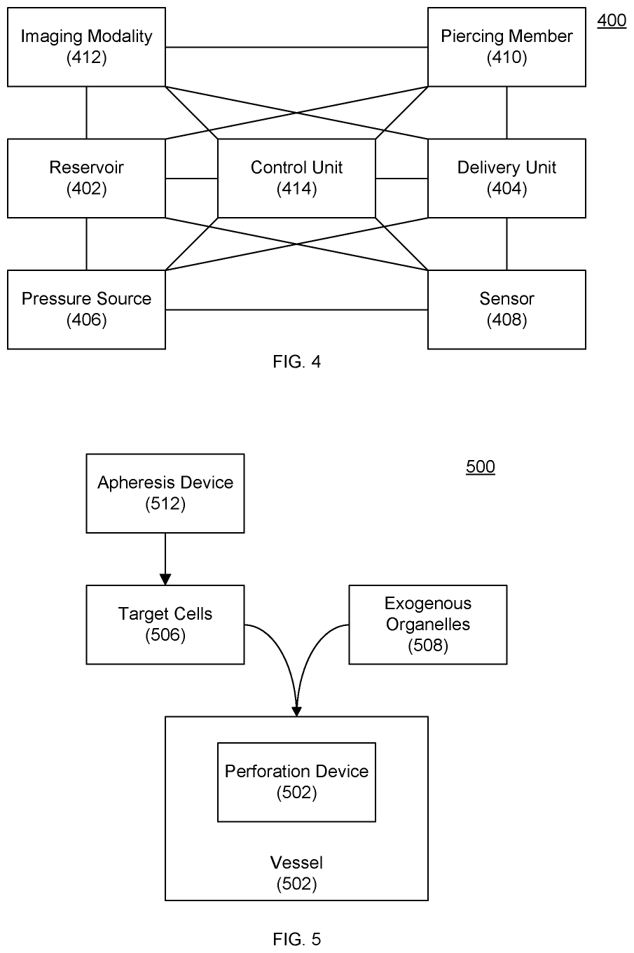

Referring now to FIG. 4, one embodiment of the invention provides a delivery device 400 including a reservoir 402 and a delivery unit 404. The delivery unit 404 can be in communication with (e.g., fluid communication through coupling to) the reservoir 402 such that the material to be delivered passes from the reservoir 402 through the delivery unit 404 and exits into or proximate to the desired location (e.g., within or adjacent to a target cell).

The reservoir 402 can include any vessel capable of holding a fluid. In some embodiments, the reservoir is closed to the atmosphere, except for through the delivery unit 404. Exemplary reservoirs 402 include syringes, tanks, pouches, bladders, and the like. In one embodiment, the reservoir 402 has a volumetric capacity between about 0.5 mL and about 500 mL, and any value in between.

Delivery unit 404 can include any vessel capable of conveying a fluid. For example, delivery unit 404 can include one or more needles (e.g., having sizes between about 7 gauge and about 34 gauge, and any value in between), cannulae, microneedles, pipettes, and the like adapted to contact, pierce and/or pass through a cell membrane.

Delivery device 400 can further include a pressure source 406. Exemplary pressure sources 406 include plungers such as used in syringes, springs, pumps, mechanical actuators, electrical actuators, electromechanical actuators (e.g., motors, servomotors), pressurized tanks or cartridges, and the like. In some embodiments, the pressure source 406 acts directly on the reservoir 402 (e.g., by compressing or increasing pressure within the reservoir 402). In other embodiments, the pressure source 406 acts indirectly on the reservoir 402 (e.g., by inducing flow in the delivery unit to draw a fluid out of the reservoir 402 (e.g., through the Venturi effect or actuation of a pump positioned along delivery unit 404).

Delivery device 400 can further include one or more sensors 408 that can be configured to assess a condition of a cell and/or the delivery device 400. For example, the sensor 408 can include a temperature sensor configured to measure a temperature of the cell and/or the delivery device 400. The sensor 408 can provide feedback regarding the positioning of delivery device 400. For example, a location of a plunger (e.g., as measured through an optical sensor and/or control of a servomotor) can be utilized to deliver a desired amount of a substance.

Delivery device 400 can further include a piercing member 410 adapted and configured to pierce a cell membrane. The piercing member 400 can be mounted on, adjacent to, or integral with the delivery unit 404. The piercing member 410 can pierce the cell membrane mechanically, such as with a blade or a beveled edge. The piercing member 410 can pierce the cell membrane thermally, e.g., through selective heating of the cell membrane or selective heating adjacent to the cell membrane that causes cavitation bubbles that, in turn, disrupt the cell membrane. Such a thermal piercing member can include a metal thin film tip that is heated using laser light as described in U.S. Patent Application Publication No. 2041/0417648 and Ting-Hsiang Wu et al., “Mitochondrial Transfer by Photothermal Nanoblade Restores Metabolite Profile in Mammalian Cells,” 23(5) Cell Metabolism 921-29 (2016).

Delivery device 400 can also include one or more imaging modalities 412 adapted and configured to facilitate placement of delivery unit 404 in a desired location. For example, various microscopes can capture the relative position of the delivery unit 404 relative to the cell.

Delivery device 400 can also include one or more controller 414. The controller 414 can be integrated within the same unit as other components 402, 404, 406, 408, 410, 412. In another embodiment, one or more controllers 414 can be external to other components 402, 404, 406, 408, 410, 412 (and sometimes an additional controller 414) and communicate with the other components 402, 404, 406, 408, 410, 412, 414 via one or more wired or wireless communication technologies.

Controller 414 can include a processor device (or central processing unit “CPU”), a memory device, a storage device, a user interface, a system bus, and/or a communication interface.

The controller 414 can, thus, provide for executing processes, by itself and/or in cooperation with one or more additional devices, that can include algorithms for controlling various components delivery device 400 in accordance with the present invention. Controller 414 can be programmed or instructed to perform these processes according to any communication protocol and/or programming language on any platform. Thus, the processes can be embodied in data as well as instructions stored in a memory device and/or storage device or received at a user interface and/or communication interface for execution on a processor.

The controller 408 can control the operation of the system components in a variety of ways. For example, controller 408 can modulate the level of electricity provided to a component. Alternatively, the controller 408 can transmit instructions and/or parameters a system component for implementation by the system component.

### Perforation and Diffusion Devices

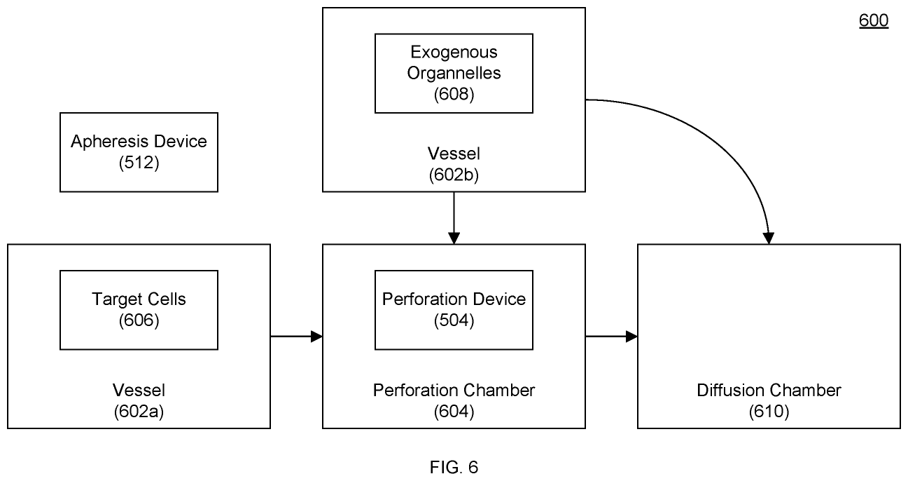

Referring now to FIGS. 5 and 6, other embodiments of the invention perforate a cell membrane and then rely on diffusion of organelles into the perforated cells.

Referring to FIG. 5, one embodiment of the invention provides a single vessel 502 housing a perforation device 504. Target cells 506 and exogenous organelles 508 can be added to the vessel 502 either prior to perforation of the cells or sequentially, in which the target cells 506 are introduced and perforated before the exogenous organelles 508 are introduced. As discussed herein, target cells can isolated using centrifugation, apheresis 512, microfluidic flow devices (e.g., those including posts) as described in as described in Daniel R. Gossett et al., “Label-free cell separation and sorting in microfluidic systems”, 397 Anal. Bioanal. Chem. 3249-67 (2010), and the like.

Referring to FIG. 6, another embodiment of the invention initially houses target cells 606 and exogenous organelles 608 in separate vessels 602a, 602b. The target cells 606 first perforated in perforation chamber 604. The exogenous organelles 608 can be introduced into the perforation chamber 604 or downstream, e.g., in a diffusion chamber 610.

A variety of perforation devices 504 and perforation chambers 604 can be used. In some embodiments, the perforation devices 504 can be electroporation electrodes, lasers, laser-induced cavitation bubbles, and the like. In some embodiments, the perforation chamber 604 achieves perforation through a flow restriction that perturbs the cell membrane as described in U.S. Patent Application Publication No. 2014/0287509 or through boundary-layer flow turbulence as described in U.S. Pat. No. 6,653,089.

### Exemplary Target Cells

As discussed herein, the devices and methods described herein can be applied to a variety of cells. Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, hepatocytes, and the like.

Without being bound by theory, Applicant believes that embodiments of the invention can be applied to a variety of cell types used for cell therapy. Exemplary cells used in cell therapy are described in publications such as N. Pavo et al., “Cell therapy for human ischemic heart diseases: critical review and summary of the clinical experiences”, 72 J. Mol. Cell. Cardiol. 12-24 (October 2014); E. Negroni et al., “Invited review: Stem cells and muscle diseases: advances in cell therapy strategies”, 41(3) Neuropathol. Appl. Neurobiol. 270-87 (April 2015); V. Bonnamain et al., “Neural stem/progenitor cells as a promising candidate for regenerative therapy of the central nervous system”, 6 Front Cell Neurosci. 17 (2012); and J. T. Daniels et al., “Limbal epithelial stem cell therapy”, 7(1) Expert Opin. Biol. Ther. 1-3 (January 2007).

### Exemplary Administration Sites

As discussed herein, the devices and methods described herein can be applied to a variety of administration sites. In one embodiment, the devices and methods facilitate parenteral administration of a therapeutic.

As used herein, “parenteral administration” of a therapeutic includes any route of administration characterized by physical breaching of a tissue of a subject and administration of the pharmaceutical composition through the breach in the tissue. Parenteral administration thus includes, but is not limited to, administration by injection of a composition, by application of the composition through a surgical incision, by application of the composition through a tissue-penetrating non-surgical wound, and the like. In particular, parenteral administration is contemplated to include, but is not limited to, subcutaneous, intravenous, intra-peritoneal, intramuscular, intrahepatic (e.g., hepatic artery, portal vein, or ductal administration), intraosseal (e.g., intrasternal), intrathecal, intracerebral, or intracerebroventricular injection, and kidney dialytic infusion techniques.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### PROPHETIC EXAMPLES

### Example 1: Double-Barrel Syringe Injection

In this example, subcellular components are loaded into a sterile polytetrafluoroethylene (PTFE) syringe, 1 mL in total volume. The syringe is attached to a hypodermic stainless steel needle, 26 gauge in diameter.

The solution is injected into the skin (subcutaneously), into muscle, into a vein or artery, into a lymph node, or into an organ tissue. In some circumstances, target tissues are exposed using surgical techniques.

In some circumstances, the syringe also includes a tissue stabilizing attachment as described in U.S. Patent Application Publication No. 2004/0082837. In such an example, subcellular components are injected directly into myocardial tissue as an adjunctive procedure during off-pump coronary artery bypass grafting. In this case, the syringe includes a toothed clip, analogous to forceps. By engaging the clip, tissue is held steady, facilitating injection through the needle.

In another example, a device delivers subcellular components to joints as a treatment of arthritic conditions, following arthroscopic surgery, or following open surgery of the knee. The device includes a 20-gauge needle syringe connected to a double barrel reservoir, one for fluid aspiration and the other for housing the subcellular components. The 20-gauge needle is advanced through the skin, muscle, and synovial capsule, penetrating the joint space. Synovial fluid is aspirated into one barrel of the syringe to remove inflammatory effusive fluid, and to confirm accurate positioning within the knee joint. The subcellular components are then injected into the joint through the second barrel. An exemplary double-barrel syringe enabling aspiration followed by injection is described in U.S. Patent Application Publication No. 2015/0112248 and U.S. Pat. No. 9,022,971.

In some circumstances, the injection device also includes an ultrasound guidance device. An ultrasound transducer and probe are connected to the needle to visualize key anatomy such as bones and vascular structures. The needle is visualized as it is advanced into the tissue for real-time guidance and feedback to ensure accurate placement.

For instance, an ultrasound-guided approach is utilized to deliver subcellular components into the ovary. In this case, an ultrasound probe is positioned within the vaginal canal. A 3″ spinal needle is mounted on a syringe housing the subcellular components. Under regional anesthesia, the needle is advanced through the wall of the vaginal canal and through subcutaneous tissue to penetrate the ovary. Subcellular components are subsequently injected into the space.

### Example 2: Autoinjection

In this example, subcellular components are loaded into a sterile, single-use autoinjection pen, and automatically injected subcutaneously into the patient.

The device includes an outer housing, an inner reservoir with the subcellular components, a needle for injection, and an actuator button. The outer housing is cylindrical in shape, 15 cm in length and 1 cm in diameter and has a clear window and indicator to monitor administration. Within the outer housing, an inner reservoir is made of PTFE, and is mounted with a needle on one side and plunger on the other side. The inner reservoir is filled with subcellular components prior to injection. The reservoir is 1 mL in volume. Pressing the actuator button moves the needle to the subcutaneous region of the skin. An internal spring then depresses the syringe plunger to inject the subcellular components subcutaneously.

The syringe is positioned at a 45-degree angle to the skin, and the patient presses the actuator button to deliver the subcellular components. The injection takes place over approximately 10 seconds, and a visual indicator on the device confirms that the full therapeutic volume has been administered.

Exemplary autoinjectors are described in U.S. Pat. No. 8,747,357.

### Example 3: Visual Aids for Syringe Injection

In this example, subcellular components are loaded into an injection device aided by a fiber optic camera and a luminal laser to visualize vocal cords.

The vocal cords are infolded mucous membrane tissues covering the larynx, that vibrate during speech. The injection device of either Example 1 or 2 is equipped with a fiber optic camera (1 mm in diameter) that is inserted into the trachea using a laryngoscope. Subcellular components are loaded into the syringe and injected through a trans-cricothyroid membrane approach. A 25 g needle is bent to a 45-degree angle, then inserted below the inferior border of the thyroid cartilage, 3 mm lateral to midline. The needle is advanced into the midline of the infraglottis, and the subcellular components are injected deep into the vocal cords.

In some cases, the device also has a luminal laser to improve visualization of the tip of the needle. The laser is connected to a three-way valve between the syringe and the hypodermic needle. Activating the laser illuminates the tissue at the tip of the hypodermic needle, allowing assessment of the needle's position through the laryngoscope camera prior to injection.

Exemplary laryngoscopes and devices for visualizing the vocal cords are described in International Publication Nos. WO 2009/025843 and WO 2010/091440.

### Example 4: Epidural Delivery Pump

In this example, subcellular components are delivered to a patient through an implantable device. The subcellular components are stored in a plastic refillable reservoir of the implantable device. An electronic signal is sent to a metering unit that resides on the bottom of the reservoir and is electronically coupled to a pump. After receiving the electronic signal, a measured volume of the subcellular components is pumped from the reservoir through a polytetrafluoroethylene (PTFE) catheter to the delivery site via the electronic pump.

The electronic pump has an electronic receiver that receives the delivery information (e.g., electronic signals) from an external programmable processor. Delivery information is entered into the programmable processor that resides outside the patient's body and transmits the electronic signals via an infrared signal to the electronic receiver in the implant.

The device is implanted into the patient subcutaneously, with the catheter inserted into the epidural space of the spine. The epidural space is identified using loss of resistance technique with a Tuohy needle and fluoroscopic imaging. The catheter is subsequently advanced into the epidural space at the level of S2-S3, a potential space created by tissue layers of the spine. The device is placed beneath the skin, and connected to the catheter prior to closure of the skin incision. After implantation, the device is activated regularly or on demand for delivery of the subcellular components to the epidural space.

In alternative examples, the catheter is placed in different areas of the body: subcutaneous, intrathecal, within an organ, or within the vascular system.

An exemplary implantable pump is described in U.S. Patent Application Publication No. 2005/0222628.

### Example 5: Encapsulated Cell Implant Delivered, Intraocular Delivery

In this example, subcellular components are enclosed within a device, then implanted into the patient. The device includes a semi-permeable membrane that allows for therapeutic delivery of macromolecules, but prevents or limits that inflammatory response from the host immune system to subcellular components.

The delivery device includes a hollow fiber fabricated from polyether sulfone with an outside diameter of 720 μm and a wall thickness of approximately 100 μm. The polyether sulfone material has a pore diameter ranging from 0.2-2 μm. This allows for passage of fluid and subcellular components, but excludes larger objects to prevent release of the subcellular components. One end of the fiber is sealed with a light-cured methacrylate resin. Subcellular components, approximately 1.5 μL, are loaded into the fiber using a temporary septal port. After liquid infusion, the tube is sealed using a methacrylate resin.

The device is loaded into a syringe-like delivery system, such as described in Example 1, mounted with a 28 gauge needle. The syringe has a plunger, which expels the device into the target tissue. In some circumstances, a tether composed of non-absorbable suture is included for retrieval of the device.

In this example, the device is implanted into the vitreous humor of the eye. Incisions are made through the conjunctiva, Tenon's capsule, and the sclera, accessing the vitreous cavity. The device is injected into the cavity to provide sustained therapeutic delivery. The vitreous cavity is subsequently closed with sutures.

Exemplary intraocular delivery devices are described in U.S. Pat. No. 9,421,129, U.S. Patent Application Publication No. 2003/0185892, and Nahid Haghjou et al., “Sustained Release Intraocular Drug Delivery Devices for Treatment of Uveitis”, 6(4) J. Ophthalmic & Vision Res. 317-29 (2011).

### Example 6: Implantable Osmotic Pump

This example describes the sustained delivery of subcellular components for 30 days in vivo using an implantable, osmotically-driven pump such as described in U.S. Pat. No. 7,207,982. The implant includes a small, cylindrical capsule sealed at one end by a flow moderator. The implant has an inner reservoir with a subcellular component-containing fluid, an osmotic layer, and a semipermeable outer membrane. Osmotic movement of body fluids into the osmotic layer compresses the internal reservoir. As a result, the pump provides steady, continuous flow of the subcellular component-containing fluid into the body.

The outer surface of the device is composed of stainless steel. The inner reservoir is made of a collapsible balloon-like silicone material, 0.9 mm in diameter and 4-5 mm in length. The permeable outer layer is composed of a non-degradable, hydrophilic, durable material, uncrosslinked hydroxylalkyl methacrylate. The flow moderator is unidirectional flap-like valve allowing subcellular components to escape, but preventing inflow of bodily fluids.

The device is implanted subcutaneously. An incision approximately 2 cm in length is made into the skin of the upper arm or leg. Subcutaneous tissue is dissected bluntly to create room for the implant to the left or right of the incision. The device is subsequently placed under the skin, and skin layers are sutured closed. Fluid is delivered to the body at a flow rate of 0.001 mL/hour.

### Example 7: Coronary Bypass Circulator

In this example, an apparatus is used for therapeutic infusion into the cardiovascular system. The apparatus is used to temporarily reproduce the effects of the cardiovascular system during coronary artery bypass grafting. The apparatus includes three units: the pump, the oxygenator, and the therapeutic storage device, controlled electronically. The circuit is connected with polyvinyl chloride (PVC) tubing. Exemplary coronary bypass circulator systems are described in U.S. Pat. No. 5,011,469.

A venous catheter is inserted into the femoral vein, and an arterial catheter is inserted into the femoral artery. These catheters are made from ethylene-vinyl-acetate (EVA) tubing. The catheter is 70 cm in length, with a diameter of less than 0.165. Both connections are sealed with suture to prevent leakage. The venous catheter is in fluid communication with the arterial pump, which provides a negative pressure to draw venous blood into the arterial pump and to draw blood out of the left ventricle of the heart. The arterial pump forces the blood through an oxygenator/heat exchange and an arterial filter.

The circuit is primed with lactated Ringer's Solution, a crystalloid fluid, prior to connection with the patient. The patient's bloodstream is fully anticoagulated using heparin to prevent formation of clots within the system. Blood flow within the device is powered by a centrifugal pump. The pump circulates blood continuously at a rate of 50 mL/min. The pump is equipped with a negative controlled to prevent pressures above 200 mmHg.

The oxygenator adds oxygen to the arterial blood and removes carbon dioxide from venous blood. The apparatus has a fluid heat exchanger to control the temperature and viscosity of the fluids within the circuit. This device also has a reservoir trap to prevent air bubble formation in the blood.

The therapeutic storage device has a reservoir to house subcellular components and is attached to a pump. The reservoir is in turn connected with the blood flow circuit, allowing for the subcellular components to be introduced into the bloodstream. The subcellular components are infused into the blood flow loop in Lactated Ringer's solution.

In an alternative example, subcellular components are housed within a semipermeable membrane as described in Example 5. The membrane is included in a cartridge spliced in line with the blood flow loop. Thus, as blood flows through the cartridge, and subcellular components release factors/compounds into the blood, without directly entering the blood stream or interacting with the host immune system.

### Example 8: Microneedle Delivery Device

In this example, subcellular components are delivered subcutaneously using a device incorporating an array of microneedles. The array of microscopic needles is applied to the skin, creating access to subcutaneous tissues. Subcellular components are infused into the body through these needles.

25 stainless steel microneedles are arranged in a square pattern, 1 cm² in area. Manufactured through microfabrication methods, these needles are cone shaped, 1500 microns in length, 1000 microns in diameter at the base, and approximately 100 microns at the tip. The cone-shaped needles are positioned perpendicular to the square array to facilitate intradermal injection. Fluid containing subcellular components is loaded into a polymer reservoir superior to the needles, closed with a cap. The reservoir is deformable, but provides stable, leak-free storage, prevents light degradation, and prevents infiltration of oxygen.

The microneedle array is pressed firmly into the skin. Delivery of the subcellular components is initiated by mechanical pressure on the top of the reservoir. The force ruptures the reservoir, allowing fluid to pass through the needles into the subcutaneous space. The fluid is rapidly absorbed in a reproducible fashion. Mechanical feedback from the device confirms that the full volume of fluid has been administered. Following delivery, the device is removed from the skin.

Exemplary microneedle devices are described in U.S. Pat. No. 6,611,707 and are available from 3M Drug Delivery Systems of Northridge, Calif.

### Example 9: Transdermal Delivery

In this example, subcellular components are delivered percutaneously through an adhesive delivery device with a reservoir for delivery through the skin. The adhesive delivery device provides continuous, sustained delivery of the subcellular components into the dermal and hypodermal layers of the skin.

The delivery device has multiple layers of components: an adhesive layer, a membrane to control the release of the fluid over time, the subcellular components are enclosed in a reservoir, a matrix filler, and a backing.

The adhesive layer attaches the device to the skin and provides a uniform surface for fluid release. This layer is composed of polyisobutylene, 1000 kDa, and a mineral oil plasticizer at a concentration of 5%. Adjacent to the adhesive layer is a subcellular component-containing reservoir. Subcellular components are suspended in phosphate-buffered saline. The two layers are separated by a semi-permeable membrane, which controls transfer to the skin. An adjacent layer has a polyurethane matrix filler to provide stiffness without directly contacting the subcellular components. The device is sealed with a backing layer composed of metal foil to prevent interaction with the external environment. The device is 5 cm in length, 5 cm in width, and approximately 5 mm in thickness.

Exemplary transdermal patches are described in U.S. Pat. No. 5,948,433 and a variety of liners, backings, membranes, and tapes for transdermal patches are available from 3M Drug Delivery Systems of Northridge, Calif.

In one example, the skin is conditioned using an abrasion tool that removes the epidermal layer. The device is applied physically to the abraded skin and left in place to facilitate sustained delivery. Various abrasion techniques and tools such as microdermabrasion and sandpaper are described in Mark R. Prausnitz & Robert Langer, “Transdermal drug delivery”, 26(11) Nat. Biotechnol. 1261-68 (2008).

### Example 10: Augmented Transdermal Delivery Through Tissue Freezing and Vacuum Pressure

This example replaces the skin abrasion step in Example 9 to facilitate transdermal delivery of subcellular components by changes in temperature and the application of vacuum pressure. By partially freezing the skin, then applying suction pressure, mechanical deformations/cracks form in the stratum corneum of the epidermis. Therapeutic application to these deformations allows for subcutaneous delivery.

The device includes a channel to apply a cooling fluid and a suction cup that covers the target skin area. Using an electromechanical pump, chlorodifluoromethane is passed through the channel onto the skin temporarily at a temperature of −26° C. for approximately 50 milliseconds, as controlled by the pump. This results in immediate cooling of the skin to −15° C. Other cooling devices include thermoelectric (Peltier) coolers, adiabatic cooling devices, fluid-cooled units that communicate with an external heat exchanger, and cryogenic devices that utilize cooled gases such as nitrogen or carbon dioxide to produce the desired low temperatures.

Immediately following fluid administration, the suction cup is applied to the skin, and vacuum is applied at 20 pounds per square inch, stretching the skin and creating cracks in the epidermis.

Cracks in the skin are approximately 100 μm in length and extend through the stratum corneum.

Exemplary methods and systems for cold and vacuum perforation of the stratum corneum are provided in U.S. Patent Application Publication No. 2011/0178456.

### Example 11: Delivery Using a Microchip Array Implant

In this example, subcellular components are delivered into the body using an implanted microchip array. Subcellular components are loaded into a sealed reservoir, which is regulated to open with a wireless remote. This results in delivery of subcellular components on a pre-determined schedule.

The microchip array is 17×17 mm, 310 μm in thickness, and contains 34 reservoirs in a uniform array. Each reservoir is loaded with a subcellular component solution, 34 μL in volume. The array is fabricated from silicon wafers using microelectronic methods: ultraviolet photolithography, chemical vapor deposition, electron beam evaporation, and reactive ion etching. The reservoirs are square pyramidal in shape and loaded with an aqueous solution of subcellular components fluid using a microsyringe pump. The reservoirs are sealed on the small square end (50×50 mm) by a 0.3-mm-thick, gold membrane anode and a silicon mating chip. Circuit traces, connecting the reservoirs to internal electronics, provided the path for a current pulse to ablate individual membranes and to expose their reservoir's contents to tissue fluid surrounding the device. After sealing, the device is sterilized using ethylene oxide gas.

The microchip array is connected to a programming device operating in the Medical Implant Communication Service (MICS) band. This wirelessly transmits instructions, such as dose scheduling, to the implant. The bidirectional communications link permits the upload of implant status information, such as dose delivery confirmation and battery voltage.

The implant location is the subcutaneous space of the abdomen, just below the waistline. Patients are given injections of lidocaine as a local anesthetic. A 2.5-cm-long incision is made through the dermis followed by blunt dissection to create a pocket of equal size to the device. Each device is placed in the pocket with the microchip facing the muscle fascia, and anchored with two suture loops to minimize micromotion in the subcutaneous space. The incision is approximated with a nylon suture.

Delivery is achieved by opening the reservoir, through the means of a wireless remote control. The remote control triggers a telemetry signal, providing a 1.04 volt potential to a specific reservoir. This allows for on-demand delivery of a bolus to the body.

### Example 12: Delivery by Inhalation

In this example, subcellular components are delivered to the patient using an inhalation device. The device includes an outer housing, a pressurized reservoir containing the subcellular components, saline, and a propellant, and a hand-operated plunger.

Subcellular components are dispersed in phosphate-buffered saline and a propellant, hydrofluoroalkane, then loaded into a pressure-resistant container. The container is fitted with a metering valve to ensure uniform dose administration. The device also has a hand operated plunger to dispense the dose. The inner surface of the plunger is linked to the metering valve, and a spring bias holds the valve in the charged position until forced to the discharge position. Actuation of the metering valve allows a metered portion of the canister content to be released, whereby the pressure of the liquefied propellant carries the subcellular components out of the container and to the patient. A valve actuator also functions to direct the aerosol as a spray into the patient's oropharynx.

Exemplary inhaler devices are described in U.S. Pat. Nos. 6,116,234 and 8,807,131.

### Example 13: Minimally Invasive Delivery to Tissue Using an Intravascular Catheter

In this example, subcellular components are injected into cardiac tissue through the use of a minimally invasive catheter with multiple needles for tissue injection.

The injection device is a hollow-tube catheter, 140 cm in length, with a luminal diameter of 1 mm. The lumen is loaded over the guidewire to ensure accurate navigation to the site of interest, and injection of the subcellular components. The distal tip of the catheter is mounted with a flexible material, such as polyether block amide polymer, to maintain shape within the blood vessel. The distal tip also has a stiff, retractable sheath, that is withdrawn mechanically by depressing a switch at the proximal end of the catheter. Retraction of the sheath exposes an array of hypodermic nitinol needles. The needles are 1 mm in length, and curve outward from the central catheter. Thus, the operator advances the needles into the tissue, and withdraws the needles by advancing the outer sheath distally using the same proximal switch. Exemplary injection devices are described in U.S. Pat. No. 6,796,963.

During the procedure, the patient's blood is anticoagulated using a bolus injection of heparin (10,000 units). A small incision is made in the groin region to access the femoral artery, which is subsequently cannulated with an access catheter. A contrast agent, such as iodine, is injected into the artery. Simultaneously, continuous X-rays are captured to visualize arterial, venous, and cardiac tissue with an intravenous contrast agent (angiography). Next, a stainless steel guidewire catheter is advanced through the femoral and iliac arteries to the abdominal aorta, and subsequently to the heart. The guidewire is positioned in the left anterior descending artery.

After placing the needles in the desired tissue, subcellular components are deposited into the tissue using a distal syringe or pump at the proximal end of the catheter. After therapeutic delivery, the needles are withdrawn and the catheter is removed. If necessary, a balloon catheter is advanced to the site of injection and inflated temporarily to aid physical contact between the subcellular components and the injection location.

In an alternative example, the device is used to deliver subcellular components to other tissues or organs within the body. For instance, subcellular components may be delivered to hepatic tissue through an intravascular approach with this device.

### Example 14: Delivery to Arterial Wall in Combination with Repair of an Arterial Occlusion

This example describes a device for local permeabilization of arteries, allowing for therapeutic delivery of subcellular components through an intravascular approach. The device is further useful for eliminating occlusions of the arterial wall.

The device is a catheter, 140 cm in length, with a hollow lumen. The device is advanced over a guidewire to ensure accurate placement. The distal end of the device has an angioplasty balloon with four small blades attached to its surface, oriented parallel to the blood vessel and positioned orthogonally along the balloon cross-section. The blades are composed of 304V stainless steel, 1 cm in length, and 0.5 mm in height. The balloon is composed of polyetherimide, and when inflated has a diameter of 6 mm.

Exemplary catheters are described in U.S. Pat. No. 5,196,024 and U.S. Patent Application Publication No. 2010/0274271.

During the procedure, the patient's blood is anticoagulated using a bolus injection of heparin (10,000 units). A small incision is made in the groin region to access the femoral artery, which is subsequently cannulated with an access catheter. A contrast agent, such as iodine, is injected into the artery. Simultaneously, continuous X-ray images are captured to visualize arterial, venous, and cardiac tissue with an intravenous contrast agent (angiography). The stainless steel guidewire catheter is advanced through the femoral and iliac arteries to the abdominal aorta, and then contralaterally to the occluded femoral artery

The device is advanced to the occluded region and inflated using a pneumatic inflation apparatus. The inflation of the balloon expands the vessel, compressing the lesion to remove the intravascular occlusion. Simultaneously, the cutting surface permeabilizes the arterial wall at the same location, enabling delivery of subcellular components through the arterial wall. Alternatively, the device is coated with subcellular components prior to implantation.

### Example 15: Automated Delivery to Deep Brain Regions

In this example, subcellular components are infused into the brain using a specialized infusion device. The device includes an implantable infusion pump and a polyurethane delivery catheter implanted surgically in the brain.

The electronic pump has an electronic receiver that receives the delivery information, e.g., electronic signals, from a programmable processor. The delivery information is entered into the programmable processor by a physician. The pump exists outside the patient's body, and the catheter is implanted into the brain. A small incision is made in the skin to reach the skull; e.g., the brain is accessed by drilling a 14 mm diameter hole in the bone. The probe is inserted stereotactically into the ventrointermedia nucleus of the thalamus. MRI guidance is used for direct visualization of brain tissue and the catheter. After implantation, the device is activated regularly or on demand for delivery of subcellular components to the brain.

Exemplary deep brain drug delivery systems are described in U.S. Pat. No. 8,412,332. Additionally, the implantable drug pump described in U.S. Patent Application Publication No. 2005/0222628 can be adapted for deep brain applications.

### Example 16: Intraosseous Infusion Using a Needle Array

In this example, subcellular components are infused into the vascular space through bone, using a device having an array of needles. The device includes an introducer needle array, a reservoir, and an infusion plunger. The reservoir is a closed space, 50 mL in volume, or alternatively connected to an external reservoir, such as a 500 mL fluid bag housing the subcellular components. To access the vascular space, the device is inserted into the sternum, 15 mm below the sternal notch. Using physical pressure, the needle array penetrates soft tissue and bone, to access vascularized bone marrow tissue. Needles are 18 gauge in diameter and one cm in length. The plunger is then depressed, infusing the subcellular components into bone marrow. The plunger injects fluid at a rate of 250 mL/min.

Exemplary intraosseous infusion devices are described in U.S. Pat. No. 5,312,364.

### Example 17: Intraaural Delivery

In this example, a delivery device is utilized to administer subcellular components to the aural space. The device includes a reservoir of subcellular components in an aqueous solution, a handle, and a nozzle. The handle has a pump to enable delivery of the solution at a pressure of up to 2000 psi, creating a needle-free, jet-based injection. The nozzle is two inches in length and 0.6 inches in diameter.

The distal end of the device is micromachined from stainless steel, and contains an array of 20 micronozzles 50 μm in diameter. The nozzles are found within the liquid reservoir of subcellular components, 500 μL in total volume. Each nozzle receives a pushrod piston able to slide longitudinally within the nozzle. The individual pistons are connected to a single plunger. The plunger is connected to a propulsion chamber with a pyrotechnic charge. Activation of the pyrotechnic charge creates a controlled combustion, advancing the pistons rapidly, and dispensing a jet of solution. The rapid injection propels subcellular components through the tympanic membrane, delivering the solution to the intraaural space and Eustachian tube.

Exemplary intraaural delivery devices are described in U.S. Patent Application Publication Nos. 2007/0055199 and 2010/0106134.

### Example 18: Device for Delivery to the Bladder

In this example, subcellular components are delivered to the bladder using a reservoir-based medical device. The device includes a dual-lumen platinum-cured silicone tube, which has a solution of subcellular components in one core and a superelastic nitinol wire, 0.23 mm in diameter, in the other core. The tube is 0.51 mm in diameter and is shaped into a pretzel-like confirmation, 35 mm in length along the major axis. The subcellular component-containing lumen is mounted with a valve to allow for selective, sustained release of a solution as a function of time.

The device is delivered to the bladder through a non-surgical cytoscopic procedure. A urethral catheter, 3.3 mm in diameter, is inserted using standard metrics to provide access to the bladder. The device is inserted into the urethral catheter, thereby temporarily straightening the device. A pusher rod is used to advance the device into the bladder. After advancing the device into the bladder, the wire bends back into its original pretzel-like shape. Because the bladder is a “storage organ,” systemic exposure of subcellular components is limited, allowing for sustained, localized delivery.

Exemplary therapeutic-releasing bladder implants are described in U.S. Pat. No. 9,114,111.

### Example 19: Delivery to the Paranasal Sinuses

Subcellular component delivery to paranasal sinuses is advantageous to clear sinus ostia. The example describes a device for targeted delivery of subcellular components to the maxillary, ethmoid, frontal, or sphenoid sinuses.

The device includes a handle and a hollow, cylindrical hypotube with a slightly curved tip. The tip is further modified with an atraumatic bulb that is bendable in different angles to facilitate entry into different sinus spaces. The tip further has an inflatable balloon, similar in function to an endovascular angioplasty balloon. When inflated using a simple water-filled syringe, the balloon is 6 mm in diameter and 20 mm in length.

Exemplary sinus balloon dilation catheters are described in U.S. Patent Application Publication No. 2013/0072958.

To deliver subcellular components, the device is navigated into the ostia of the target sinus. The balloon is inflated, creating a temporarily closed cavity. The subcellular components are injected into the cavity through the internal lumen and allowed to bathe the walls of the sinus. Subcellular components are trapped within mucous along the walls of the sinus. Subsequently, fluid is aspirated from the cavity, the balloon is deflated, and the device is withdrawn.

### Example 20: Intraluminal Injection Via Endoscopy

This example describes the injection of subcellular components into the luminal wall of the gastrointestinal (GI) tract using an endoscope. It is applicable either to upper GI (accessed orally) or the lower GI (accessed rectally). The endoscope has a handle and an insertion tube. The insertion tube has a moveable distal tip which is controlled electronically at the handle. Additionally, the endoscope is mounted with a fiber optic light source and video camera to provide direct feedback to the operator.

Exemplary endoscopes are described in U.S. Patent Application Publication No. 2009/0198212.

The endoscope is navigated to the site of interest, such as a peptic ulcer. An injection catheter is advanced through the lumen of the endoscope to reach the target site. The injection catheter is one meter in length and 2.8 mm in outer diameter, and 0.5 mm in inner diameter. The needle is slightly recessed within the catheter, allowing for navigation without damaging the endoscope or tissue. The 23 g needle is deployed by depressing a mechanical actuator and extends approximately 2 cm beyond the end of the catheter. An adjustable positive stop prevents the needle from advancing too far into target tissues. This allows full penetration into the tissue. Subcellular components are injected into the tissue using a syringe connected to the proximal end of the injection catheter, then the needle is retracted by reversing the actuator.

### Example 21: Device for Intrauterine Delivery

In this example, subcellular components are delivered into the uterus using a device that is advanced into the uterus through the vaginal canal, and subcellular components are sprayed into the space as droplets.

The device includes a catheter, a handle, a reservoir, and an actuating plunger. Subcellular components are placed in a liquid solution and loaded into a HDPE reservoir. The reservoir is connected to the plunger on one side and the catheter on the other side. The catheter is 15 cm in length and made of semi-rigid polyethylene. The tip of the catheter is mounted with a spray nozzle. As liquid is forced out of the tip, it forms small droplets containing subcellular components. The droplets are sprayed outward in a cone shape.

Exemplary in vitro fertilization devices that can be utilized to apply a therapeutic are described in U.S. Patent Application Publication No. 2014/0316214.

The cervix is visualized using a retractor, and gently grasped using ring forceps. The tip of the device is advanced into the uterine fundus. Depression of the plunger deploys the subcellular components. The droplet mechanism ensures uniform coverage of the uterine walls.

### Example 22: Ultrasound-Guided Injection in Utero into Amniotic Fluid for Fetal Delivery

In this example, subcellular components are delivered to a fetus using a transabdominal device.

The device includes a fetoscope for direct visualization of the fetal tissue, as well as a sheath that passes the surgical instruments, such as catheters, into the amniotic fluid. The fetoscope incorporates a 30 cm long, 3 mm diameter hypotube trocar, composed of stainless steel. The top of the tube is mounted with an eyepiece to allow visualization with a fiber optic cable. The hypotube also includes a sheath and valve for insertion of an aspiration catheter or an injection catheter.

Fetoscopes are available from KARL STORZ Endoscopy-America, Inc. of El Segundo, Calif.

Under local anesthesia, a small laparotomy is made by incising the skin, fat, and muscle to expose the uterine wall. The procedure is conducted under ultrasound guidance to visualize the needle tip, ensure placement within amniotic fluid, and avoid harm to fetal tissue. The needle of the fetoscope is advanced through the uterine wall into amniotic fluid. The subcellular components are injected into the amniotic fluid within the uterus. After injection, the catheter and the device are withdrawn. Subcutaneous fascia and skin incisions are subsequently closed using standard techniques.

In an alternative example, the subcellular components are injected directly into fetal tissue. In this case, ultrasound guidance is used for placement of a 28 gauge needle catheter into the fetal tissue. A syringe is mounted on the proximal end of the catheter, enabling injection of up to 1 mL of fluid.

### Example 23: Intraventricular Delivery

In this example, subcellular components are delivered to the brain using an intraventricular delivery device such an intraventricular catheter.

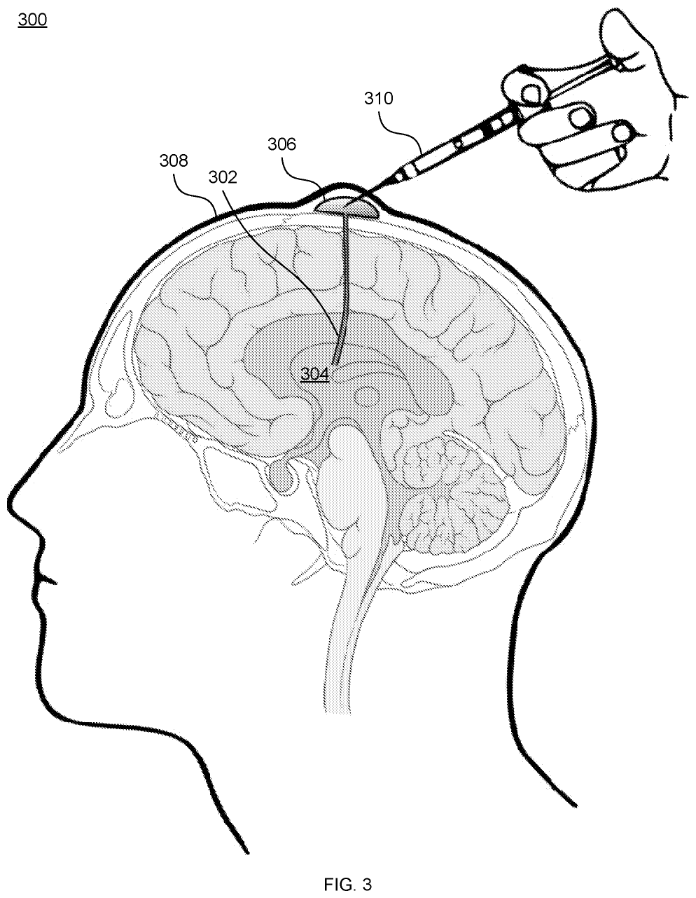

One example of an intraventricular catheter is the Ommaya reservoir 300 depicted in FIG. 3. The Ommaya reservoir 300 includes a catheter 302 that can be positioned in a lateral ventricle 304. The catheter 302 is coupled to a reservoir 306 that can be implanted under the scalp 308. A material to be administered can be periodically introduced to the reservoir 306, e.g., through injection with a syringe 310.

### Example 24: Intracellular Transfer Using a Nanoblade Device

The example describes a device for precise transfer of subcellular components into cells. The device includes a functionalized micron-sized tip, connected with a light source, a pressure source, and subcellular component solution. Activating the device when contacting a cell results in a transient permeation of the cell membrane through a cavitation bubble, as well as injection of the subcellular components. The procedure is visualized microscopically.

The device includes a hollow, micron-sized tip, 3 mm in diameter and 5 mm long. A 100 nm titanium thin film is deposited on the tip using a sputterer deposition system.

Exemplary devices are described in U.S. Patent Application Publication No. 2011/0117648 and Ting-Hsiang Wu et al., “Mitochondrial Transfer by Photothermal Nanoblade Restores Metabolite Profile in Mammalian Cells,” 23(5) Cell Metabolism 921-29 (2016).

The tip is connected to an external pressure source and a 532 nm nanosecond pulsed laser. A 403 0.6 NA objective lens is used to view target cell-tip placement and to channel a pulsed laser beam onto the sample plane. The laser is a Q-switched, frequency-doubled Nd:YAG laser with a linearly polarized laser pulse output at 532 nm in wavelength and 6 ns in pulse width. A half wave plate and a polarizing beam splitter are installed in the laser beam path to adjust the laser energy. An optical diffuser and two convex lenses, f1=25 mm and f2=60 mm, are placed in the beam path to smooth out the laser intensity profile and to control the dimension of the laser spot size on the sample/imaging plane. The laser spot diameter at the imaging plane is 260 mm.

A programmable pressure source drives the subcellular components into the cell through a separate channel. A double pole, single throw switch is used to coordinately trigger laser pulsing and subcellular component delivery activities.

The light source beam is aligned into the epi-fluorescence port of the microscope and reflected into the back aperture of the objective lens onto the sample plane by a dichroic mirror. A longpass filter is used to block any back-scattered light from reaching an imaging camera. A motorized micromanipulator is mounted to the device to enable accurate positioning.

The injection pressure is set to 15 hPa, and injection time is 0.1 s to minimize cell lysis from the delivered fluid volume. Resuspended subcellular components (0.5 mg/ml protein concentration) are kept on ice until delivery. 8 μl of isolated subcellular components are loaded into the device. The device tip is positioned to lightly contact the target cell surface. The device is activated by depressing the switch, resulting in a simultaneous laser pulse and delivery of donor subcellular components.

### Example 25: High Throughput Delivery Using an Electric Field

This example describes a device enabling high throughput delivery of subcellular components to target cells using an electric field. The device includes a surface with cells, a light/energy source, and a solution of subcellular components. The device includes an optical energy source that heats a fluid medium through a porous membrane. This forms nanoscale, rapidly expanding cavitation bubbles. The hydrodynamic and mechanical forces exerted by the bubbles transiently open cells for delivery of the subcellular components.

The device includes a circular reservoir with a porous polymer membrane platform, and titanium side walls, 1 cm in diameter. The membrane is composed of polyester covered in a micron-scale titanium thin film. Using a laser ablation at an oblique angle, the film is removed from the surface, but maintained within the pores of the polyester. The membrane is functionalized with approximately 106 pores, lined with titanium, and 3 μm in diameter.

The main reservoir is connected to two source chambers by tubing and a pump. The first chamber houses the target cells; the second chamber holds the donor subcellular components.

Electronic activation pumps the contents from both chambers into the reservoir.

A laser is positioned directly beneath the polymer membrane platform. An in-line lens applies the laser energy uniformly over the entire surface of the membrane.

An exemplary device is described in U.S. Patent Application Publication No. 2016/0017340.

Cells are deposited into the reservoir in a solution of phosphate buffered saline. The laser is pulsed at 532 nm for 6 ns, resulting in an energy transfer of 113.2 mJ/cm². Cavitation bubbles are produced that flow through the mesh and disturb the cell membranes. The subcellular components are then transferred from the second chamber and allowed to mix with the permeabilized cells within the reservoir. Based on diffusion, the subcellular components travel into the permeabilized cell membranes.

In an alternative embodiment, polymorphonuclear cells are isolated directly from the blood, permeated and supplemented with subcellular components, then returned to the blood stream using an apheresis device. This example utilizes a similar procedure as described herein. However, the chamber housing the target cells is fed by an apheresis device, and the main reservoir is connected to the apheresis circuit.

The apheresis device includes a venous access catheter, a separation device, and a venous return device. Blood is taken from the patient, separated into component parts, and ‘unused’ components, such as erythrocytes, platelets, and plasma, are returned to the patient. This enables delivery of extracorporeal subcellular components to a portion of blood cells without requiring large volumes of a patient's blood.

### Example 26: Flow-Based Transfer in Microfluidic Channels

This example describes a microfluidic-based device that physically disrupts target cellular membranes, creating a temporary period of permeability due to pressure and shear stress. During this period, the target cell is immersed in a solution of subcellular components, which flow into the target cell based on concentration gradient.

The device employs a series of ceramic microfluidic channels with a small diameter, such that cells are constricted when flowing through the channels. The channels are tubular, 50 μm in length, 5-6 μm in (smaller than that of a cell). The target cells are suspended in phosphate buffered saline and housed in a reservoir. The reservoir is attached to a pump, which forces the target cells through the channels at 500 mm/s.

An exemplary device is described in U.S. Patent Application Publication No. 2014/0287509.

After passing through the channels, permeabilized cells are collected in a secondary reservoir. A concentrated solution of subcellular components is pumped into the secondary reservoir. Subcellular components move down the concentration gradient by diffusing into the target cells. After approximately 20 minutes of incubation, cell membrane permeability decreases and the subcellular components are fully transferred.

### Example 27: Delivery Enabled by an Electroporation Reservoir Device

In this example, target cell membranes are temporarily permeabilized using an electroporation device. During this period of permeability, subcellular components are transferred through diffusion.

The device includes a stainless steel reservoir with electrodes connected to a high-voltage electrical power source.

Exemplary electroporation devices are available under the MICROPULSER™ mark from Bio-Rad Laboratories, Inc. of Hercules, Calif. and are described, for example, in U.S. Pat. No. 7,799,555.

Target cells are washed and resuspended in glycerol, then placed in the reservoir. The reservoir is conditioned to 37° C. using a fluid-bath warmer. The device provides a rapid, transient pulse of electrical current through the solution. The pulse is administered at 1,000 volts for three milliseconds using a 10 microfarad capacitor in parallel with a 600 ohm resistor.

After electroporation, a concentrated solution of subcellular components is added to the reservoir. The subcellular components spontaneously diffuse into the target cells. After approximately 60 minutes of incubation, cell membrane permeability decreases and the subcellular components are fully transferred.

### Example 28: Subcellular Components Delivery to Many Membrane Enclosed Objects

This example describes a device to selectively implant subcellular components into a target cell.

The device includes a microfluidic fluidic channel combined with a substance that promotes permeation of the cell membrane. The device has three input reservoirs connected to a microfluidic channel that flow into an output reservoir. The reservoirs are fabricated from PETE and connected to channels by PVC tubing that flow into a collection reservoir. Each of the channels is 300 μm in width and 30 μm in depth.

Microfluidic channels are fabricated using a silicon-PDMS casting method. Capillary channels are micromachined on a silanized silicon water using a CAD-based photoresist method. A negative relief of poly(dimethylsiloxane) (PDMS) is formed by curing a prepolymer over the silicon mold. Finally, a glass cover plate is bonded to the top surface of the PDMS, resulting in closed channels.

An exemplary device is described in U.S. Pat. No. 6,653,089.

Target cells are placed in one reservoir, subcellular components are placed in the second reservoir, and a membrane-permeable actin-disrupting drug, latrunculin, is placed in the third channel. The three channels come together to simultaneously permeabilize the cell membrane and encourage infiltration of the subcellular components past the cell membrane. The fluid flow rate in the main channel is 0.6 cm/s. Thus, cells flow through the device and are supplemented with subcellular components before reaching the collection reservoir.

In an alternate usage of the device, flow within the channels is modulated, such that latrunculin acts on only a portion of the cell membrane. After permeabilizing a portion of the cell membrane, subcellular components freely flow into the cell as both fluid streams pass through the channel.

### Example 29: Direct Cell Implantation to Oocytes Under Optical Guidance (In Vitro Addition of Subcellular Components to an Egg)

This example describes a device that transfers subcellular components to an oocyte under direct microscopic guidance. The device includes an electroporator to fuse donor subcellular components with the target oocyte, an imaging system attached to an inverted microscope with a drill micromanipulator having a 6 μm inner diameter microcapillary end, and a micropipette with a 20-25 μm outer diameter. The micromanipulator stabilizes the oocyte for injection with the micropipette.

An exemplary device is described in U.S. Patent Application Publication No. 2012/0036591.

Target oocytes are transferred to 30 μL manipulation droplets of TH3 with 5 μg/ml cytochalasin B on a glass bottom manipulation dish covered with paraffin oil (Zander IVF) and incubated at 37° C. for 10-15 minutes. Subcellular components are injected close to the left end of the oocyte where the oocyte is held with the micromanipulator. A gentle aspiration is applied to the oocyte to aspirate a small amount of cytoplasm and the injected subcellular components into the microcapillary end of the micromanipulator. A cytoplast is generated by quickly pulling the microcapillary end away from the oocyte. Two 50 μs DC pulses of 2.7 kV/cm from the electroporator induce cell fusion of the cytoplast with the oocyte. Following cell fusion, subcellular components are transferred into the oocyte.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### APPENDIX

### Definitions

As used herein, a “cell membrane” refers to a membrane derived from a cell, e.g., a source cell or a target cell.

As used herein, a “chondrisome” is a subcellular apparatus derived and isolated or purified from the mitochondrial network of a natural cell or tissue source. A “chondrisome preparation” has bioactivity (can interact with, or have an effect on, a cell or tissue) and/or pharmaceutical activity.

As used herein, a chondrisome preparation described herein is “stable” when it maintains a predefined threshold level of its activity and structure over a defined period of time. In some embodiments, one or more (2 or more, 3 or more, 4 or more, 5 or more) structural and/or functional characteristics of a chondrisome preparation described can be used as defining metrics of stability for chondrisome preparations described herein. These metrics, whose assay protocols are outlined herein, are determined subsequent to preparation and prior to storage (e.g., at 4 C, 0 C, −4 C, −20 C, −80 C) and following removal from storage. The characteristic of the preparation should not change by more than 95%, 90%, 85%, 80%, 75%, 60%, 50% (e.g., no more than 40%, 35%, 30%, 25%, 20%, 15%, 10%, 5%) over the course of 1, 2, 5, 8, 12, 24, 36, or 48 hours, 3 days, 7 days, 14 days, 21 days, 30 days, 60 days, 90 days, 4 months, 6 months, 9 months, a year or more of storage. In some embodiments, the characteristic of the chondrisome preparation described herein should not have changed by more than 50% (e.g., no more than 40%, 35%, 30%, 25%, 20%, 15%, 10%, 5%) over the course of 1, 2, 5, 8, 12, 24, 36, or 48 hours of storage. In some embodiments, the characteristic of the chondrisome preparation described herein should not change by more than 50% (e.g., no more than 40%, 35%, 30%, 25%, 20%, 15%, 10%, 5%) over the course of 1, 2, 5, 8, 12, 24, 36, or 48 hours, 3 days, 7 days, 14 days, 21 days, 30 days, 60 days, 90 days, 4 months, 6 months, 9 months, a year or more of storage.

As used herein, “cytobiologic” refers to a portion of a cell that comprises a lumen and a cell membrane, or a cell having partial or complete nuclear inactivation. In some embodiments, the cytobiologic comprises one or more of a cytoskeleton component, an organelle, and a ribosome. In embodiments, the cytobiologic is an enucleated cell, a microvesicle, or a cell ghost.

As used herein, “cytosol” refers to the aqueous component of the cytoplasm of a cell. The cytosol may comprise proteins, RNA, metabolites, and ions.

An “exogenous agent” as used herein, refers to an agent that: i) does not naturally exist, such as a protein that has a sequence that is altered (e.g., by insertion, deletion, or substitution) relative to an endogenous protein, or ii) does not naturally occur in the naturally occurring source cell of the fusosome in which the exogenous agent is disposed.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., the lumen of the fusosome and a cytoplasm of a target cell).

As used herein, “fusogen binding partner” refers to an agent or molecule that interacts with a fusogen to facilitate fusion between two membranes.

As used herein, “fusosome” refers to a membrane enclosed preparation and a fusogen that interacts with the amphipathic lipid bilayer.

As used herein, “fusosome composition” refers to a composition comprising one or more fusosomes.

As used herein, “locally” or “local administration” means administration at a particular site of the body intended for a local effect. Examples of local administration include epicutaneous, inhalational, intra-articular, intrathecal, intravaginal, intravitreal, intrauterine, intra-lesional administration, lymph node administration, intratumoral administration, administration to a fat tissue or mucous membrane of the subject, wherein the administration is intended to have a local effect. Local administration may also include perfusion of the preparation into a target tissue. For example, a preparation described herein may be delivered locally to the cardiac tissue (i.e., myocardium, pericardium, or endocardium) by direct intracoronary injection, or by standard percutaneous catheter based methods or by perfusion into the cardiac tissue. In another example, the preparation is infused into the brain or cerebrospinal fluid using standard methods. In another example, the preparation is directly injected into adipose tissue of a subject.

As used herein, “membrane enclosed preparation” refers to a bilayer of amphipathic lipids enclosing a cargo in a lumen or cavity. In some embodiments, the cargo is exogenous to the lumen or cavity. In other embodiments, the cargo is endogenous to the lumen or cavity, e.g., endogenous to a source cell.

As used herein, “mitochondrial biogenesis” denotes the process of increasing biomass of mitochondria. Mitochondrial biogenesis includes increasing the number and/or size of mitochondria in a cell.

As used herein, the term “purified” means altered or removed from the natural state. For example, a cell or cell fragment naturally present in a living animal is not “purified,” but the same cell or cell fragment partially or completely separated from the coexisting materials of its natural state is “purified.” A purified fusosome composition can exist in substantially pure form, or can exist in a non-native environment such as, for example, a culture medium such as a culture medium comprising cells.

As used herein, a “source cell” refers to a cell from which a fusosome is derived.

As used herein, a “subcellular component” is a subcellular apparatus derived and isolated or purified from a natural cell or tissue source.

### Fusosomes

In some aspects, the fusosome compositions and methods described herein comprise membrane enclosed preparations, e.g., naturally derived or engineered lipid membranes, comprising a fusogen. In some aspects, the disclosure provides a portion of a non-plant cell, e.g., a mammalian cell, or derivative thereof (e.g., a mitochondrion, a chondrisome, an organelle, or an enucleated cell), which comprises a fusogen, e.g., protein, lipid and chemical fusogens.

### Fusogens

In some embodiments, the fusosome described herein (e.g., a liposome, a vesicle, a portion of a cell) includes one or more fusogens, e.g., to facilitate the fusion of the fusosome to a membrane, e.g., a cell membrane. Also, these compositions may include surface modifications made during or after synthesis to include one or more fusogens, e.g., fusogens may be complementary to a target cell.

In some embodiments, the fusosomes comprise one or more fusogens on their exterior surface to target a specific cell or tissue type (e.g., cardiomyocytes). Fusogens include, without limitation, protein based, lipid based, and chemical based fusogens. The fusogen may bind a partner on a target cells' surface. In some embodiments, the fusosome comprising the fusogen will integrate the membrane into a lipid bilayer of a target cell.

In some embodiments, one or more of the fusogens described herein may be included in the fusosome.

### Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein, a native protein or a derivative of a native protein, a synthetic protein, a fragment thereof, a protein fusion comprising one or more of the fusogens or fragments, and any combination thereof.

### Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi: 10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties (see Table 3), a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

### Non-Mammalian Proteins

In some embodiments, the fusogen may include a non-mammalian protein, e.g., a viral protein. In some embodiments, a viral fusogen is a Class I viral membrane fusion protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV).

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devce1.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

### Other Proteins

In some embodiments, the fusogen may include a pH dependent (e.g., as in cases of ischemic injury) protein, a homologue thereof, a fragment thereof, and a protein fusion comprising one or more proteins or fragments thereof. Fusogens may mediate membrane fusion at the cell surface or in an endosome or in another cell-membrane bound space.

In some embodiments, the fusogen includes a EFF-1, AFF-1, gap junction protein, e.g., a connexin (such as Cn43, GAP43, CX43) (DOI: 10.1021/jacs.6b05191), other tumor connection proteins, a homologue thereof, a fragment thereof, a variant thereof, and a protein fusion comprising one or more proteins or fragments thereof

### Lipid Fusogens

In some embodiments, the fusogen is a fusogenic lipid, such as saturated fatty acid. In some embodiments, the saturated fatty acids have between 10-14 carbons. In some embodiments, the saturated fatty acids have longer-chain carboxylic acids. In some embodiments, the saturated fatty acids are mono-esters.

In some embodiments, the fusosome may be treated with unsaturated fatty acids. In some embodiments, the unsaturated fatty acids have between C16 and C18 unsaturated fatty acids. In some embodiments, the unsaturated fatty acids include oleic acid, glycerol mono-oleate, glycerides, diacylglycerol, modified unsaturated fatty acids, and any combination thereof.

Without wishing to be bound by theory, in some embodiments negative curvature lipids promote membrane fusion. In some embodiments, the fusosome comprises one or more negative curvature lipids, e.g., exogenous negative curvature lipids, in the membrane. In embodiments, the negative curvature lipid or a precursor thereof is added to media comprising source cells or fusosomes. In embodiments, the source cell is engineered to express or overexpress one or more lipid synthesis genes. The negative curvature lipid can be, e.g., diacylglycerol (DAG), cholesterol, phosphatidic acid (PA), phosphatidylethanolamine (PE), or fatty acid (FA).

Without wishing to be bound by theory, in some embodiments positive curvature lipids inhibit membrane fusion. In some embodiments, the fusosome comprises reduced levels of one or more positive curvature lipids, e.g., exogenous positive curvature lipids, in the membrane. In embodiments, the levels are reduced by inhibiting synthesis of the lipid, e.g., by knockout or knockdown of a lipid synthesis gene, in the source cell. The positive curvature lipid can be, e.g., lysophosphatidylcholine (LPC), phosphatidylinositol (PtdIns), lysophosphatidic acid (LPA), lysophosphatidylethanolamine (LPE), or monoacylglycerol (MAG).

### Chemical Fusogens

In some embodiments, the fusosome may be treated with fusogenic chemicals. In some embodiments, the fusogenic chemical is polyethylene glycol (PEG) or derivatives thereof.

In some embodiments, the chemical fusogen induces a local dehydration between the two membranes that leads to unfavorable molecular packing of the bilayer. In some embodiments, the chemical fusogen induces dehydration of an area near the lipid bilayer, causing displacement of aqueous molecules between cells and allowing interaction between the two membranes together.

In some embodiments, the chemical fusogen is a positive cation. Some nonlimiting examples of positive cations include Ca2+, Mg2+, Mn2+, Zn2+, La3+, Sr3+, and H+.

In some embodiments, the chemical fusogen binds to the target membrane by modifying surface polarity, which alters the hydration-dependent intermembrane repulsion.

In some embodiments, the chemical fusogen is a soluble lipid soluble. Some nonlimiting examples include oleoylglycerol, dioleoylglycerol, trioleoylglycerol, and variants and derivatives thereof.

In some embodiments, the chemical fusogen is a water-soluble chemical. Some nonlimiting examples include polyethylene glycol, dimethyl sulphoxide, and variants and derivatives thereof.

In some embodiments, the chemical fusogen is a small organic molecule. A nonlimiting example includes n-hexyl bromide.

In some embodiments, the chemical fusogen does not alter the constitution, cell viability, or the ion transport properties of the fusogen or target membrane.

In some embodiments, the chemical fusogen is a hormone or a vitamin. Some nonlimiting examples include abscisic acid, retinol (vitamin A1), a tocopherol (vitamin E), and variants and derivatives thereof.

In some embodiments, the fusosome comprises actin and an agent that stabilizes polymerized actin. Without wishing to be bound by theory, stabilized actin in a fusosome can promote fusion with a target cell. In embodiments, the agent that stabilizes polymerized actin is chosen from actin, myosin, biotin-streptavidin, ATP, neuronal Wiskott-Aldrich syndrome protein (N-WASP), or formin. See, e.g., Langmuir. 2011 Aug. 16; 27(16):10061-71 and Wen et al., Nat Commun. 2016 Aug. 31; 7. In embodiments, the fusosome comprises exogenous actin, e.g., wild-type actin or actin comprising a mutation that promotes polymerization. In embodiments, the fusosome comprises ATP or phosphocreatine, e.g., exogenous ATP or phosphocreatine.

### Small Molecule Fusogens

In some embodiments, the fusosome may be treated with fusogenic small molecules. Some nonlimiting examples include halothane, nonsteroidal anti-inflammatory drugs (NSAIDs) such as meloxicam, piroxicam, tenoxicam, and chlorpromazine.

In some embodiments, the small molecule fusogen may be present in micelle-like aggregates or free of aggregates.

### Fusosome Generation

Fusosomes Generated from Cells

Compositions of fusosomes may be generated from cells in culture, for example cultured mammalian cells, e.g., cultured human cells. The cells may be progenitor cells or non-progenitor (e.g., differentiated) cells. The cells may be primary cells or cell lines (e.g., a mammalian, e.g., human, cell line described herein). In embodiments, the cultured cells are progenitor cells, e.g., bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

The cultured cells may be from epithelial, connective, muscular, or nervous tissue or cells, and combinations thereof. Fusosome can be generated from cultured cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)′; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In embodiments, the cells are from a highly mitotic tissue (e.g., a highly mitotic healthy tissue, such as epithelium, embryonic tissue, bone marrow, intestinal crypts). In embodiments, the tissue sample is a highly metabolic tissue (e.g., skeletal tissue, neural tissue, cardiomyocytes).

A fusosome composition described herein may be comprised of fusosomes from one cellular or tissue source, or from a combination of sources. For example, a fusosome composition may comprise fusosomes from xenogeneic sources (e.g. animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise fusosomes in different metabolic states, e.g. coupled or uncoupled, as described elsewhere herein.

In some embodiments, fusosomes are generated by inducing budding of a mitoparticle, pyrenocyte, exosome, liposome, lysosome, or other membrane enclosed vesicle.

In some embodiments, fusosomes are generated by inducing cell enucleation. Removing the nucleus of a cell may be performed using assays known in the art, such as genetic, chemical, mechanical methods, or combinations thereof. Enucleation refers not only to a complete removal of the nucleus but also the displacement of the nucleus from its typical location such that the cell contains the nucleus but it is non-functional.

In some embodiments, fusosomes are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

### Synthetic Fusosomes

Certain components of synthetic fusosomes may be generated from a cell or a tissue, for example, the fusogen, the lipid, or the cargo. In some embodiments, the fusogen may be derived from xenogeneic sources (e.g., animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise synthetic fusosomes in different metabolic states, e.g. coupled or uncoupled, as described elsewhere herein.

Additional production techniques useful for making synthetic fusosomes, e.g., filter based vesicle production/alteration of size distribution, are described in Spuch and Navarro, Journal of Drug Delivery, vol. 2011, Article ID 469679, 12 pages, 2011. doi:10.1155/2011/469679 and Templeton et al., Nature Biotech, 15:647-652, 1997.

### Cargo

In some aspects, the disclosure provides a composition (e.g., a pharmaceutical composition) comprising (i) one or more of a chondrisome (e.g., as described in international application, PCT/US16/64251), a mitochondrion, an organelle (e.g., Mitochondria, Lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granuole, and networks of organelles), or an enucleated cell, e.g., an enucleated cell comprising any of the foregoing, and (ii) a fusogen, e.g., a myomaker protein. In embodiments, the fusogen is present in a lipid bilayer external to the mitochondrion or chondrisome. In embodiments, the chondrisome has one or more of the properties as described, for example, in international application, PCT/US16/64251.

In some embodiments, the cargo may include one or more nucleic acid sequences, one or more polypeptides, a combination of nucleic acid sequences and/or polypeptides, one or more organelles, and any combination thereof. In some embodiments, the cargo may include one or more cellular components. In some embodiments, the cargo includes one or more cytosolic and/or nuclear components.

In some embodiments, the cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof.

In some embodiments, the cargo may include a nucleic acid. For example, RNA to enhance expression of an endogenous protein, or a siRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the cargo is a nucleic acid that targets a transcriptional activator that modulate structure or function in the target cells.

In some embodiments, the cargo includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, DNA integration polypeptides, targeted endonucleases (e.g. Zinc-finger nucleases, transcription-activator-like nucleases (TALENs), cas9 and homologs thereof), recombinases, and any combination thereof.

In some embodiments, the cargo includes a small molecule, e.g., ions (e.g. Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof.

In some embodiments, the cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granuole, networks of organelles, and any combination thereof.

In one aspect, the fusosome, e.g., a pharmaceutical composition of, or a composition of, comprises isolated chondrisomes (e.g., a chondrisome preparation), derived from a cellular source of mitochondria.

In another aspect, the fusosome, e.g., a pharmaceutical composition of, or a composition of, comprises isolated, modified chondrisomes (e.g., modified chondrisome preparation) derived from a cellular source of mitochondria.

In another aspect, the fusosome, e.g., a pharmaceutical composition of, or a composition of, comprises chondrisomes (e.g., chondrisome preparation) expressing an exogenous protein.

### Delivery

In certain aspects, the disclosure provides a method of delivering a membrane enclosed preparation to a target cell in a subject. In some embodiments, the method comprises administering to a subject a fusosome, e.g., a membrane enclosed preparation comprising a nucleic acid encoding a fusogen, e.g., a myomaker protein, wherein the nucleic acid is not within a cell, under conditions that allow the fusogen to be expressed on the surface of the fusosome in the subject. In some embodiments, the method further comprises administering to the subject a composition comprising an agent, e.g., a therapeutic agent, and a fusogen binding partner, optionally, comprising a carrier, e.g., a membrane, under conditions that allow fusion of the fusogen on the fusosome and the fusogen binding partner. In some embodiments, the carrier comprises a membrane, e.g., a lipid bilayer, e.g., the agent is disposed within a lipid bilayer. In some embodiments, the lipid bilayer fuses with the target cell, thereby delivering the agent to the target cell in the subject.

In some embodiments, the fusogen on a fusosome interacts with a fusogen binding partner on target membrane to induce fusion of between the fusosome and the target membrane. In some embodiments, the fusogen interacts with a fusogen binding partner on subcellular organelles, including mitochondria.

In some embodiments, a fusogen (e.g., protein, lipid or chemical fusogen) or a fusogen binding partner is delivered to a target cell or tissue prior to, at the same time, or after the delivery of a fusosome.

In some embodiments, a fusogen (e.g., protein, lipid or chemical fusogen) or a fusogen binding partner is delivered to a non-target cell or tissue prior to, at the same time, or after the delivery of a fusosome.

In some embodiments, a nucleic acid that encodes a fusogen (e.g., protein or lipid fusogen) or a fusogen binding partner is delivered to a target cell or tissue prior to, at the same time, or after the delivery of a fusosome.

In some embodiments, a polypeptide, nucleic acid, ribonucleoprotein, or small-molecule that upregulates or downregulates expression of a fusogen (e.g., protein, lipid or chemical fusogen) or a fusogen binding partner is delivered to a target cell or tissue prior to, at the same time, or after the delivery of a fusosome.

In some embodiments, a polypeptide, nucleic acid, ribonucleoprotein, or small-molecule that upregulates or downregulates expression of a fusogen (e.g., protein, lipid or chemical fusogen) or a fusogen binding partner is delivered to a non-target cell or tissue prior to, at the same time, or after the delivery of a fusosome.

In some embodiments, the target cell or tissue is modified by (e.g. inducing stress or cell division) to increase the rate of fusion prior to, at the same time, or after the delivery of a fusosome. Some nonlimiting examples include, inducing ischemia, treatment with a chemotherapy, antibiotic, irradiation, toxin, inflammation, inflammatory molecules, anti-inflammatory molecules, acid injury, basic injury, burn, polyethylene glycol, neurotransmitters, myelotoxic drugs, growth factors, or hormones, tissue resection, starvation, and/or exercise.

In some embodiments, the target cells or tissue is treated with an epigenetic modifier, e.g., a small molecule epigenetic modifier, to increase or decrease expression of an endogenous cell surface molecule, e.g., a fusogen binding partner, e.g., an organ, tissue, or cell targeting molecule, where the cell surface molecule is a protein, glycan, lipid or low molecular weight molecule.

In some embodiments, the target cell or tissue is treated with a vasodilator (e.g. nitric oxide (NO), carbon monoxide, prostacyclin (PGI2), nitroglycerine, phentolamine) or vasoconstrictors (e.g. angiotensin (AGT), endothelin (EDN), norepinephrine)) to increase the rate of fusosome transport to the target tissue.

In some embodiments, the target cell or tissue is treated with a chemical agent, e.g., a chemotherapeutic. In such embodiments, the chemotherapeutic induces damage to the target cell or tissue that enhances fusogenic activity of target cells or tissue.

In some embodiments, the target cell or tissue is treated with a physical stress, e.g., electrofusion. In such embodiments, the physical stress destabilizes the membranes of the target cell or tissue to enhance fusogenic activity of target cells or tissue.

In some embodiments, the target cell or tissue may be treated with an agent to enhance fusion with a fusosome. For example, specific neuronal receptors may be stimulated with an anti-depressant to enhance fusogenic properties.

Compositions comprising the fusosomes described herein may be administered or targeted to the circulatory system, hepatic system, renal system, cardio-pulmonary system, central nervous system, peripheral nervous system, musculoskeletal system, lymphatic system, immune system, sensory nervous systems (sight, hearing, smell, touch, taste), digestive system, endocrine systems (including adipose tissue metabolic regulation), reproduction system.

In embodiments, a fusosome composition described herein is delivered ex-vivo to a cell or tissue, e.g., a human cell or tissue. In some embodiments, the composition is delivered to an ex vivo tissue that is in an injured state (e.g., from trauma, disease, hypoxia, ischemia or other damage).

In some embodiments, the fusosome composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). The composition improves viability, respiration, or other function of the transplant. The composition can be delivered to the tissue or organ before, during and/or after transplantation.

The fusosome compositions described herein can be used to treat a subject, e.g., a human, in need thereof. In such embodiments, the subject may be at risk, may have a symptom of, or may be diagnosed with or identified as having, a particular disease or condition (e.g., a disease or condition described herein).

In some embodiments, the source of fusosomes are from the same subject that is treated with a fusosome composition. In other embodiments, they are different. For example, the source of fusosomes and recipient tissue may be autologous (from the same subject) or heterologous (from different subjects). In either case, the donor tissue for fusosome compositions described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

### Example A-1: Sonication-Mediated Generation of Fusosomes

This example describes loading of fusogens into a fusosome via sonication. Sonication methods are disclosed e.g., in Lamichhane, T N, et al., Oncogene Knockdown via Active Loading of Small RNAs into Extracellular Vesicles by Sonication. Cell Mol Bioeng, (2016), the entire contents of which are hereby incorporated by reference.

Fusosomes are prepared by any one of the methods described herein. Approximately 10⁶ fusosomes are mixed with 5-20 μg protein and incubated at room temperature for 30 minutes. The fusosome/protein mixture is then sonicated for 30 seconds at room temperature using a water bath sonicator (Brason model #1510R-DTH) operated at 40 kHz. The mixture is then placed on ice for one minute followed by a second round of sonication at 40 kHz for 30 seconds. The mixture is then centrifuged at 16,000 g for 5 minutes at 4 C to pellet the fusosomes containing protein. The supernatant containing unincorporated protein is removed and the pellet is resuspended in phosphate-buffered saline. After protein loading, the fusosomes are kept on ice before use.

### Example A-2: Generation of Fusosomes Through Protein Electroporation

This example describes electroporation of fusogens to generate fusosomes.

Approximately 5×10⁶ cells or vesicles are used for electroporation using an electroporation transfection system (Thermo Fisher Scientific). To set up a master mix, 24 μg of purified protein fusogens is added to resuspension buffer (provided in the kit). The mixture is incubated at room temperature for 10 min. Meanwhile, the cells or vesicles are transferred to a sterile test tube and centrifuged at 500×g for 5 min. The supernatant is aspirated and the pellet is resuspended in 1 ml of PBS without Ca²⁺ and Mg²⁺. The buffer with the fusogens is then used to resuspend the pellet of cells or vesicles. A cell or vesicle suspension is also used for optimization conditions, which vary in pulse voltage, pulse width and the number of pulses. After electroporation, the electroporated cells or vesicles with fusogens are washed with PBS, resuspended in PBS, and kept on ice.

See, for example, Liang et al., Rapid and highly efficiency mammalian cell engineering via Cas9 protein transfection, Journal of Biotechnology 208: 44-53, 2015.

### Example A-3: Generating and Isolating Giant Plasma Membrane Fusosomes

This example describes fusosome generation and isolation via vesiculation and centrifugation. This is one of the methods by which fusosomes may be isolated. Fusosomes are prepared as follows.

Briefly, HeLa cells that express a fusogen are washed twice in buffer (10 mM HEPES, 150 mM NaCl, 2 mM CaCl₂, pH 7.4), resuspended in a solution (1 mM DTT, 12.5 mM Paraformaldehyde, and 1 mM N-ethylmaleimide in GPMV buffer), and incubated at 37° C. for 1 h. Fusosomes are clarified from cells by first removing cells by centrifugation at 100×g for 10 minutes, and then harvesting fusosomes at 20,000×g for 1 h at 4° C. The fusosomes are resuspended in desired buffer for experimentation.

See for example, Sezgin E et al. Elucidating membrane structure and protein behavior using giant membrane plasma vesicles. Nat. Protocols. 7(6):1042-51 2012.

### Example A-4: Generating and Isolating Fusosome Ghosts

This example describes fusosome generation and isolation via hypotonic treatment and centrifugation. This is one of the methods by which fusosomes may be produced.

First, fusosomes are isolated from mesenchymal stem cells expressing fusogens (10⁹ cells) primarily by using hypotonic treatment such that the cell ruptures and fusosomes are formed. According to a specific embodiment, cells are resuspended in hypotonic solution, Tris-magnesium buffer (TM, e.g., pH 7.4 or pH 8.6 at 4° C., pH adjustment made with HCl). Cell swelling is monitored by phase-contrast microscopy. Once the cells swell and fusosomes are formed, the suspension is placed in a homogenizer. Typically, about 95% cell rupture is sufficient as measured through cell counting and standard AOPI staining. The membranes/fusosomes are then placed in sucrose (0.25 M or higher) for preservation. Alternatively, fusosomes can be formed by other approaches known in the art to lyse cells, such as mild sonication (Arkhiv anatomii, gistologii i embriologii; 1979, August, 77(8) 5-13; PMID: 496657), freeze-thaw (Nature. 1999, Dec. 2; 402(6761):551-5; PMID: 10591218), French-press (Methods in Enzymology, Volume 541, 2014, Pages 169-176; PMID: 24423265), needle-passaging (www.sigmaaldrich.com/technical-documents/protocols/biology/nuclear-protein-extraction.html) or solublization in detergent-containing solutions (www.thermofisher.com/order/catalog/product/89900).

To avoid adherence, the fusosomes are placed in plastic tubes and centrifuged. A laminated pellet is produced in which the topmost lighter gray lamina includes mostly fusosomes. However, the entire pellet is processed, to increase yields. Centrifugation (e.g., 3,000 rpm for 15 min at 4° C.) and washing (e.g., 20 volumes of Tris magnesium/TM-sucrose pH 7.4) may be repeated.

In the next step, the fusosome fraction is separated by floatation in a discontinuous sucrose density gradient. A small excess of supernatant is left remaining with the washed pellet, which now includes fusosomes, nuclei, and incompletely ruptured whole cells. An additional 60% w/w sucrose in TM, pH 8.6, is added to the suspension to give a reading of 45% sucrose on a refractometer. After this step, all solutions are TM pH 8.6. 15 ml of suspension are placed in SW-25.2 cellulose nitrate tubes and a discontinuous gradient is formed over the suspension by adding 15 ml layers, respectively, of 40% and 35% w/w sucrose, and then adding 5 ml of TM-sucrose (0.25 M). The samples are then centrifuged at 20,000 rpm for 10 min, 4° C. The nuclei sediment form a pellet, the incompletely ruptured whole cells are collected at the 40%-45% interface, and the fusosomes are collected at the 35%-40% interface. The fusosomes from multiple tubes are collected and pooled.

See for example, International patent publication, WO2011024172A2.

### Example A-5: Physical Enucleation of Fusosomes

This example describes enucleation of fusosomes via cytoskeletal inactivation and centrifugation. This is one of the methods by which fusosomes may be modified.

Fusosomes are isolated from mammalian primary or immortalized cell lines that express a fusogen. The cells are enucleated by treatment with an actin skeleton inhibitor and ultracentrifugation. Briefly, C2Cl2 cells are collected, pelleted, and resuspended in DMEM containing 12.5% Ficoll 400 (F2637, Sigma, St. Louis Mo.) and 500 nM Latrunculin B (ab144291, Abcam, Cambridge, Mass.) and incubated for 30 minutes at 37° C.+5% CO₂. Suspensions are carefully layered into ultracentrifuge tubes containing increasing concentrations of Ficoll 400 dissolved in DMEM (15%, 16%, 17%, 18%, 19%, 20%, 3 mL per layer) that have been equilibrated overnight at 37° C. in the presence of 5% CO₂. Ficoll gradients are spun in a Ti-70 rotor (Beckman-Coulter, Brea, Calif.) at 32,300 RPM for 60 minutes at 37 C. After ultracentrifugation, fusosomes found between 16-18% Ficoll are removed, washed with DMEM, and resuspended in DMEM.

Staining for nuclear content with Hoechst 33342 as described in Example 35 followed by the use of flow cytometry and/or imaging will be performed to confirm the ejection of the nucleus.

### Example A-6: Generating Fusosomes Through Extrusion

This example describes fusosome manufacturing by extrusion through a membrane.

Briefly, hematopoietic stem cells that express fusogens are in a 37° C. suspension at a density of 1×10⁶ cells/mL in serum-free media containing protease inhibitor cocktail (Set V, Calbiochem 539137-1ML). The cells are aspirated with a luer lock syringe and passed once through a disposable 5 mm syringe filter into a clean tube. If the membrane fouls and becomes clogged, it is set aside and a new filter is attached. After the entire cell suspension has passed through the filter, 5 mL of serum-free media is passed through all filters used in the process to wash any remaining material through the filter(s). The solution is then combined with the extruded fusosomes in the filtrate.

Fusosomes may be further reduced in size by continued extrusion following the same method with increasingly smaller filter pore sizes, ranging from 5 mm to 0.2 mm. When the final extrusion is complete, suspensions are pelleted by centrifugation (time and speed required vary by size) and resuspended in media.

Additionally, this process can be supplemented with the use of an actin cytoskeleton inhibitor in order to decrease the influence of the existing cytoskeletal structure on extrusion. Briefly, a 1×10⁶ cell/mL suspension is incubated in serum-free media with 500 nM Latrunculin B (ab144291, Abcam, Cambridge, Mass.) and incubated for 30 minutes at 37° C. in the presence of 5% CO₂. After incubation, protease inhibitor cocktail is added and cells are aspirated into a luer lock syringe, with the extrusion carried out as previously described.

Fusosomes are pelleted and washed once in PBS to remove the cytoskeleton inhibitor before being resuspended in media.

### Example A-7: Processing Fusosomes

This example described the processing of fusosomes. Fusosomes produced via any of the described methods in the previous Examples may be further processed.

In some embodiments, fusosomes are first homogenized, e.g., by sonication. For example, the sonication protocol includes a 5 second sonication using an MSE sonicator with microprobe at an amplitude setting of 8 (Instrumentation Associates, N.Y.). In some embodiments, this short period of sonication is enough to cause the plasma membrane of the fusosomes to break up into homogenously sized fusosomes. Under these conditions, organelle membranes are not disrupted and these are removed by centrifugation (3,000 rpm, 15 min 4° C.). Fusosomes are then purified by differential centrifugation as described in Example A-5.

Extrusion of fusosomes through a commercially available polycarbonate membrane (e.g., from Sterlitech, Wash.) or an asymmetric ceramic membrane (e.g., Membralox), commercially available from Pall Execia, France, is an effective method for reducing fusosome sizes to a relatively well defined size distribution. Typically, the suspension is cycled through the membrane one or more times until the desired fusosome size distribution is achieved. The fusosomes may be extruded through successively smaller pore membranes (e.g., 400 nm, 100 nm and/or 50 nm pore size) to achieve a gradual reduction in size and uniform distribution.

In some embodiments, at any step of fusosome production, though typically prior to the homogenization, sonication and/or extrusion steps, a pharmaceutical agent (such as a therapeutic), may be added to the reaction mixture such that the resultant fusosome encapsulates the pharmaceutical agent.

### Example A-8: In Vivo Delivery of Membrane Protein

This example describes fusosome fusion with a cell in vivo. In an embodiment, fusosome fusion with a cell in vivo results in delivery of an active membrane protein to the recipient cell. In this example, the fusosomes comprise the Sendai virus HVJ-E protein as in the previous Example. In an embodiment, the fusosomes are generated to comprise the membrane protein, GLUT4. Fusosomes with and without GLUT4 are prepared as described herein.

BALB/c-nu mice are administered fusosomes comprising GLUT4, fusosomes that do not comprise GLUT4, or PBS (negative control). Mice are injected intramuscularly in the tibialis anterior muscle with fusosomes or PBS. Immediately prior to fusosome administration, mice are fasted for 12 hours and injected with [18F] 2-fluoro-2deoxy-d-glucose (18F-FDG), which is an analog of glucose that enables positron emission tomography (PET imaging). Mice are injected with 18F-FDG via the tail vein under anesthesia (2% isoflurane). PET imaging is performed using a nanoscale imaging system (1T, Mediso, Hungary). Imaging is conducted 4 hours after administration of fusosomes. Immediately after imaging, mice are sacrificed and the tibialis anterior muscle is weighed. PET images are reconstructed using a 3D imaging system in full detector mode, with all corrections on, high regularization, and eight iterations. Three-dimensional volume of interest (VOI) analysis of the reconstructed images is performed using the imaging software package (Mediso, Hungary) and applying standard uptake value (SUV) analysis. VOI fixed with a diameter of 2 mm sphere, is drawn for the tibialis anterior muscle site. The SUV of each VOI sites is calculated using the following formula: SUV=(radioactivity in volume of interest, measured as Bq/cc×body weight)/injected radioactivity.

In an embodiment, mice that are administered fusosomes comprising GLUT4 will demonstrate an increased radioactive signal in VOI as compared to mice administered PBS or fusosomes that do not comprise GLUT4.

See, also, Yang et al., Advanced Materials 29, 1605604, 2017.

### Example A-9: In Vivo Delivery of Protein

This example describes the delivery of therapeutic agents to the eye by fusosomes.

Fusosomes are produced as described herein and are loaded with a protein that is deficient in a mouse knock-out.

Fusosomes are injected subretinally into the right eye of a mouse that is deficient for the protein and vehicle control is injected into the left eye of the mice. A subset of the mice is euthanized when they reach 2 months of age.

Histology and H&E staining of the harvested retinal tissue is conducted to count the number of cells rescued in each retina of the mice (described in Sanges et al., The Journal of Clinical Investigation, 126(8): 3104-3116, 2016).

The level of the injected protein is measured in retinas harvested from mice euthanized at 2 months of age via a western blot with an antibody specific to the therapeutic protein.

In an embodiment, the left eyes of mice, which are administered fusosomes, will have an increased number of nuclei present in the outer nuclear level of the retina compared to the right eyes of mice, which are treated with vehicle. The increased protein is suggestive of complementation of the mutated protein.

### Example A-10: In Vivo Delivery of DNA

This example describes the delivery of DNA to cells in vivo via fusosomes. Delivery of DNA to cells in vivo results in the expression of proteins within the recipient cell.

Fusosome DNA delivery in vivo will demonstrates the delivery of DNA and protein expression in recipient cells within an organism (mouse).

Fusosomes that express a liver directed fusogen are prepared as described herein. Following production of the fusosome, it is additionally nucleofected with a plasmid having a sequence that codes for Cre recombinase.

Fusosomes are prepared for in vivo delivery. Fusosome suspensions are subjected to centrifugation. Pellets of the fusosomes are resuspended in sterile phosphate buffered saline for injection.

Fusosomes are verified to contain DNA using a nucleic acid detection method, e.g., PCR.

The recipient mice harbor a loxp-luciferase genomic DNA locus that is modified by CRE protein made from DNA delivered by the fusosomes to unblock the expression of luciferase (JAX #005125). The positive control for this example are offspring of recipient mice mated to a mouse strain that expresses the same protein exclusively in the liver from its own genome (albumin-CRE JAX #003574). Offspring from this mating harbor one of each allele (loxp-luciferase, albumin-CRE). Negative controls are carried out by injection of recipient mice with fusosomes not expressing fusogens or fusosomes with fusogens but not containing Cre DNA.

The fusosomes are delivered into mice by intravenous (IV) tailvein administration. Mice are placed in a commercially available mouse restrainer (Harvard Apparatus). Prior to restraint, animals are warmed by placing their cage on a circulating water bath. Once inside the restrainer, the animals are allowed to acclimate. An IV catheter consisting of a 30G needle tip, a 3″ length of PE-10 tubing, and a 28G needle is prepared and flushed with heparinized saline. The tail is cleaned with a 70% alcohol prep pad. Then, the catheter needle is held with forceps and slowly introduced into the lateral tail vein until blood becomes visible in the tubing. The fusosome solution (˜500K-5M fusosomes) is aspirated into a 1 cc tuberculin syringe and connected to an infusion pump. The fusosome solution is delivered at a rate of 20 uL per minute for 30 seconds to 5 minutes, depending on the dose. Upon completion of infusion, the catheter is removed, and pressure is applied to the injection site until cessation of any bleeding. Mice are returned to their cages and allowed to recover.

After fusion, the DNA will be transcribed and translated into CRE protein which will then translocates to the nucleus to carry out recombination resulting in the constitutive expression of luciferase. Intraperitoneal administration of D-luciferin (Perkin Elmer, 150 mg/kg) enables the detection of luciferase expression via the production of bioluminescence. The animal is placed into an in vivo bioluminescent imaging chamber (Perkin Elmer) which houses a cone anesthetizer (isoflurane) to prevent animal motion. Photon collection is carried out between 8-20 minutes post-injection to observe the maximum in bioluminescence due to D-luciferin pharmacokinetic clearance. A specific region of the liver is created in the software and collection exposure time set so that count rates are above 600 (in this region) to yield interpretable radiance (photons/sec/cm2/steradians) measurements. The maximum value of bioluminescent radiance is recorded as the image of bioluminescence distribution. The liver tissue is monitored specifically for radiance measurements above background (untreated animals) and those of negative controls. Measurements are carried out at 24 hours post-injection to observe luciferase activity. Mice are then euthanized and livers are harvested.

Freshly harvested tissue is subjected to fixation and embedding via immersion in 4% paraformaldehyde/0.1M sodium phosphate buffer pH7.4 at 4° C. for 1-3 hrs. Tissue is then immersed in sterile 15% sucrose/1×PBS (3 hrs. to overnight) at 4° C. Tissue is then embedded in O.C.T. (Baxter No. M7148-4). Tissue is oriented in the block appropriately for sectioning (cross-section). Tissue is then frozen in liquid nitrogen using the following method: place the bottom third of the block into the liquid nitrogen, allow to freeze until all but the center of the O.C.T. is frozen, and allow freezing to conclude on dry ice. Blocks are sectioned by cryostat into 5-7 micron sections placed on slides and refrozen for staining.

In situ hybridization is carried out (using standard methods) on tissue sections using digoxygenin labeled nucleic acid probes (for CRE DNA and luciferase mRNA detection), labeled by anti-digoxygenin fluorescent antibodies, and observed by confocal microscopy.

In embodiments, positive control animals (recombination via breeding without fusosome injection) will show bioluminescence intensity in liver as compared to untreated animals (no CRE and no fusosomes) and negative controls, while agent injected animals will show bioluminescence in liver as compared to negative controls (fusosomes without fusogen) and untreated animals.

In embodiments, detection of nucleic acid in tissue sections in agent injected animals will reveal detection of CRE recombinase and luciferase mRNA compared to negative controls and untreated animals in cells in the tissue, while positive controls will show levels of both luciferase mRNA and CRE recombinase DNA throughout the tissue.

Evidence of DNA delivery by fusosomes will be detected by in situ hybridization-based detection of the DNA and its colocalization in the recipient tissue of the animal. Activity of the protein expressed from the DNA will be detected by bioluminescent imaging. In embodiments, fusosomes will deliver DNA that will result in protein production and activity.

### Example A-11: Delivery of Mitochondria Via Protein Enhanced Fusogenic Enucleated Cells

Fusogens are imaged on a Zeiss LSM 780 inverted confocal microscope at 63× magnification 24 h following deposition in the imaging dish. Cells expressing only Mito-DsRed alone and Mito-GFP alone are imaged separately to configure acquisition settings in such a way as to ensure no signal overlap between the two channels in conditions where both Mito-DsRed and Mito-GFP are both present and acquired simultaneously. Ten regions of interest are chosen in a completely unbiased manner, with the only criteria being that a minimum of 10 cells be contained within each ROI, such that a minimum number of cells are available for downstream analysis. A given pixel in these images is determined to be positive for mitochondria if it's intensity for either channel (mito-DsRed and mito-GFP) is greater than 10% of the maximum intensity value for each respective channel across all three ROIs.

Fusion events with organelle delivery will be identified based on the criteria that >50% of the mitochondria (identified by all pixels that are either mito-GFP+ or mito-Ds-Red+) in a cell are positive for both mitoDs-Red and mito-GFP based on the above indicated threshold, which will indicate that organelles (in this case mitochondria) containing these proteins are delivered, fused and their contents intermingled. At the 24-hour time point multiple cells are expected to exhibit positive organelle delivery via fusion.

1. A device for delivering material into a biological tissue comprising:
a reservoir for the material; and
a material delivery unit in connection with the reservoir configured to transfer the material from the reservoir to the tissue.

2. The device of claim 1, wherein the reservoir has a volumetric capacity in the range of about 0.5 mL to about 500 mL.

3. The device of claim 1, wherein the reservoir is in connection with a metering unit.

4. The device of claim 1, wherein the reservoir is in connection with a pump.

5. The device of claim 1, wherein the reservoir is configured to maintain a specific temperature, pressure, or viscosity of the material prior to delivery.

6. The device of claim 1, wherein the reservoir is configured to allow preparation of the material prior to delivery.

7. The device of claim 6, wherein the preparation comprises one or more selected from the group consisting of: mixing, temperature, and viscosity optimization for delivery.

8. The device of claim 1, wherein the reservoir is pressurized to aerosolize the material.

9. The device of claim 1, wherein the reservoir comprises a permeable membrane.

10. The device of claim 9, wherein the permeable membrane comprises one or more selected from the group consisting of: a natural polymer, a synthetic polymer, a stent with a polymer coating, and a hydrogel/polymer matrix.

11. The device of claim 9, wherein the permeable membrane has a pore size of less than about 10 μm, less than about 9 μm, less than about 8 μm, less than about 7 μm, less than about 6 μm, less than about 5 μm, less than about 4 μm, less than about 3 μm, less than about 2 μm, less than about 1 μm, less than about 500 nm, less than about 200 nm, less than about 100 nm, and less than about 50 nm.

12. The device of claim 1, wherein the material delivery unit comprises electrical circuitry configured to generate at least one selected from the group consisting of: a thermal change, a physical contact force, an ultrasonic frequency, an osmotic change, a pressure change, a photothermal pulse, a magnetic field, an electromagnetic field, an electric field, and an electrical pulse through the reservoir.

13. The device of claim 1, wherein the material delivery unit comprises a material-dispensing member that transfers the material from the reservoir to the tissue.

14. The device of claim 13, wherein the material-dispensing member is selected from the group consisting of: a metering unit and a pump.

15. The device of claim 1, wherein the material delivery unit is configured for insertion into a patient's body.

16. The device of claim 1, wherein the device is implantable or insertable into a subject.

17. The device of claim 1, wherein the material delivery unit comprises a tissue-penetrating member for piercing tissue.

18. The device of claim 17, wherein the tissue-penetrating member comprises a single injector.

19. The device of claim 17, wherein the tissue-penetrating member is configured to pierce the tissue to a preselected depth.

20. The device of claim 19, wherein the preselected depth is suitable for one or more selected from the group consisting of: transdermal, transendothelial, transepithelial, atherosclerolic, extravesicular, arteriole, venous, and peritoneal applications.

21. The device of claim 17, wherein the tissue-penetrating member comprises an array of injectors.

22. The device of claim 21, wherein the array of injectors is configured to pierce the tissue at a uniform depth or multiple depths.

23. The device of claim 21, wherein the material delivery unit comprises one or more selected from the group consisting of: a stent, tubing, a balloon, and a microneedle.

24. The device of claim 21, wherein the material delivery unit comprises a catheter fluidly connected to the reservoir.

25. The device of claim 24, wherein the catheter is configured to be removably connected to the reservoir.

26. The device of claim 24, wherein the catheter is a Peripherally Inserted Central Catheter (PICC).

27. The device of claim 24, wherein the catheter further comprises a balloon.

28. The device of claim 1, further comprising a plunger configured to expel the material out of the reservoir into the material delivery unit.

29. The device of claim 1, further comprising a tissue-conditioning apparatus.

30. The device of claim 29, wherein the material delivery unit is configured to deliver the material into the tissue upon application of the tissue-conditioning apparatus.

31. The device of claim 29, wherein the tissue-conditioning apparatus is adapted and configured to alter the tissue to increase uptake of the material within the tissue.

32. The device of claim 29, wherein the tissue-conditioning apparatus comprises a light source.

33. The device of claim 29, wherein the tissue-conditioning apparatus is configured to abrade, puncture, or thermally ablate a surface of the tissue.

34. The device of claim 29, wherein the tissue-conditioning apparatus is configured to expose the tissue to at least laser or high-frequency radio waves.

35. The device of claim 29, wherein the tissue-conditioning apparatus is configured to expose the tissue to at least one selected from the group consisting of: a thermal change, a physical contact force, a shear contact force, an ultrasonic frequency, a photothermal pulse, a magnetic field, an electromagnetic field, an electric field, and an electrical pulse.

36. The device of claim 1 further comprising an imaging device.

37. The device of claim 36, wherein the imaging device is selected from the group consisting of: a camera, an X-ray imaging detector, ultrasound, a computed tomography (CT) device, a magnetic resonance imaging (MM) device, an arthroscopic device, and an endoscope.

38. The device of claim 1, wherein the device has a largest cross-sectional profile selected from the group consisting of: less than about 10 mm², less than about 5 mm², less than about 4 mm², less than about 3 mm², less than about 2 mm², or less than about 1 mm².

39. The device of claim 1, wherein the reservoir is hermetically sealed and the material delivery unit is configured to require activation to release the material from the reservoir.

40. The device of claim 39, wherein the activation is an electric pulse.

41. The device of claim 1, further comprising a tissue stabilizer comprising a tissue contacting member, wherein the tissue stabilizer is operatively associated with the material delivery unit.

42. The device of claim 41, wherein the tissue stabilizer is adapted and configured to hold the tissue during actuation of the material delivery unit.

43. The device of claim 1, further comprising a closed-loop system.

44. The device of claim 43, wherein the closed-loop system is an apheresis device.

45. The device of claim 1, further comprising a sensor configured to obtain a measurement of the tissue.

46. The device of claim 1 further comprising a computer.

47. The device of claim 46, wherein the computer is programmed to perform one or more functions selected from the group consisting of: storing information, regulating delivery, adjusting delivery in response to a measurement, and adjusting delivery in response to a measurement from a sensor.

48. The device of claim 1, wherein the device is configured for delivery to a specific tissue type.

49. The device of claim 48, wherein the specific tissue type is selected from the group consisting of: muscle, epithelial tissue, connective tissue, and nervous tissue.

50. The device of claim 1, wherein the device is configured for delivery to a specific body location.

51. The device of claim 50, wherein the specific body location is selected from the group consisting of: cardiovasculature, circulatory system, digestive tract, excretory organs, CNS, lymph nodes, immune organs, musculoskeletal tissues, respiratory organs, reproductive organs, and a placenta.

52. An implantable or insertable delivery device for delivery of material across or into a biological tissue in a subject, the device comprising:
a reservoir for holding the material; and
a tissue-penetrating member.

53. The device of claim 52, wherein the reservoir is in connection with a metering unit.

54. The device of claim 52, wherein the reservoir is configured to maintain a specific temperature, pressure, or viscosity of the material prior to delivery.

55. The device of claim 52, wherein the reservoir is configured to perform one or more steps to the material prior to delivery, the one or more steps selected from the group consisting of: preparation, mixing, temperature optimization for delivery, and viscosity optimization for delivery.

56. The device of claim 52, further comprising a plunger configured to expel the material out of the reservoir into the tissue.

57. The device of claim 52, wherein the tissue-penetrating member is adapted and configured to pierce tissue.

58. The device of claim 52, wherein the tissue-penetrating member comprises a single injector.

59. The device of claim 52, wherein the tissue-penetrating member is configured to pierce the tissue to a preselected depth.

60. The device of claim 52, wherein the tissue-penetrating member comprises an array of injectors.

61. The device of claim 61, wherein the array of injectors pierces the tissue at a uniform depth or multiple depths.

62. The device of claim 52, wherein the tissue-penetrating member is a catheter.

63. The device of claim 62, wherein the catheter is configured to be removably connected to the reservoir.

64. The device of claim 62, wherein the catheter is a Peripherally Inserted Central Catheter (PICC).

65. A system for delivering material into a biological tissue, the system comprising:
a tissue conditioning apparatus;
a reservoir for the material; and
a material delivery unit in connection with a reservoir configured to transfer the material from the reservoir to the tissue.

66. The system of claim 65, wherein the biological tissue is skin.

67. The system of claim 65, wherein the reservoir has a volumetric capacity in the range of about 0.5 mL to about 500 mL.

68. The system of claim 65, wherein the reservoir is in connection with a metering unit.

69. The system of claim 65, wherein the reservoir is in connection with a pump.

70. The system of claim 65, wherein the reservoir is configured to maintain a specific temperature, pressure, or viscosity of the material prior to delivery.

71. The system of claim 65, wherein the reservoir is configured to perform one or more steps to the material prior to delivery, the one or more steps selected from the group consisting of: preparation, mixing, temperature optimization for delivery, and viscosity optimization for delivery.

72. The system of claim 65, wherein the tissue conditioning apparatus comprises a light source.

73. The system of claim 65, wherein the tissue conditioning apparatus is configured to abrade, puncture, or thermally ablate a surface of the tissue.

74. The system of claim 65, wherein the tissue conditioning apparatus is configured to expose the tissue to at least one selected from the group consisting of: an electric field, a magnetic field, an electromagnetic field, a photothermal energy, and an ultrasonic frequency.

75. The system of claim 65, wherein the reservoir comprises a membrane and the material delivery unit aids absorption of the material into the tissue.

76. The system of claim 76, wherein the membrane is selected from the group consisting of: a transdermal patch and a sublingual patch.

77. A delivery device for material transfer across a membrane-enclosed object comprising a reservoir and a microfluidic channel, wherein movement of the membrane-enclosed object through the microfluidic channel permeabilizes the membrane to allow movement of a material through the membrane

78. The device of claim 77, wherein the membrane-enclosed object has a maximal cross-sectional dimension selected from the group consisting of: less than 5 μm, less than 4 μm, and less than 3 μm.

79. The device of claim 77, wherein the membrane-enclosed object is selected from the group consisting of: a cell, a microparticle, a vesicle, an organelle, and an endosome.

80. The device of claim 77, wherein the microfluidic channel contacts the membrane to permeabilize the membrane.

81. The device of claim 77, wherein the microfluidic channel has a diameter of at least 10% of the maximum cross-sectional dimension of a cell.

82. The device of claim 77, wherein the membrane-enclosed object is selected from the group consisting of: a cell, a microparticle, a vesicle, an organelle, and an endosome.

83. The device of claim 77, further comprising electrical circuitry.

84. The device of claim 83, wherein the electrical circuitry is configured to generate an electrical pulse through the microfluidic channel.

85. The device of claim 83, wherein the electrical circuitry is configured to generate at least one selected from the group consisting of: a thermal change, a physical contact force, an ultrasonic frequency, an osmotic change, a pressure change, a photothermal pulse, a magnetic field, an electromagnetic field, an electric field, and an electrical pulse through the microfluidic channel.

86. The device of claim 83, wherein the electrical circuitry is configured to allow transfer of the material into the object at a specific ratio of material-to-object as measured by quantity, by mass, or by volume.

87. The device of claim 86, wherein the ratio of material-to-object is in a range of about 1:1 to about 20:1.

88. The device of claim 83, wherein the electrical circuitry is configured to maintain a specific temperature, pressure, or viscosity of the material prior to movement through the membrane.

89. The device of claim 77, further comprising: a pump configured to maintain a flow through the microfluidic channel.

90. The device of claim 77, wherein the reservoir comprises an inlet and an outlet for fluidic movement of cells into and out of the reservoir.

91. A system for material transfer into a plurality of membrane-enclosed objects comprising a reservoir and a microfluidic channel, wherein the microfluidic channel contacts a membrane of the membrane-enclosed objects to permeabilize the membrane and allow movement of a material through the membrane.

92. The system of claim 91, wherein the membrane-enclosed object is selected from the group consisting of: a cell, a microparticle, a vesicle, an organelle, and an endosome.

93. The system of claim 91, wherein the microfluidic channel has a diameter of at least 10% of the maximum cross-sectional dimension of a cell to permeabilize the membrane.

94. The system of claim 91, wherein the microfluidic channel is capable of permeabilizing at least 100 cells per minute, 1,000 cells per minute, 10,000 cells per minute, or 100,000 cells per minute.

95. The system of claim 91, wherein the system is configured to facilitate transfer of the material into the membrane-enclosed objects at a specific ratio of material-to-object as measured by quantity, by mass, or by volume.

96. The system of claim 95, wherein the ratio of material-to-object is in a range of about 1:1 to about 20:1.

97. The system of claim 91, wherein the system is configured to maintain a specific temperature, pressure, viscosity of the material prior to movement through the membrane.

98. A system for material transfer into a plurality of membrane-enclosed objects comprising a reservoir and electrical circuitry configured to generates at least one selected from the group consisting of: an electric field, a magnetic field, an electromagnetic field, a photothermal pulse, and an ultrasonic frequency in the reservoir to permeabilize a membrane of the object and allow movement of a material through the membrane.

99. The system of claim 98, wherein the electrical circuitry is configured to facilitate transfer of the material into the membrane-enclosed objects at a specific ratio of material-to-object as measured by quantity, by mass, or by volume.

100. The system of claim 99, wherein the ratio of material-to-object is in a range of about 1:1 to about 20:1.

101. The system of claim 98, wherein the electrical circuitry is configured to generate a temperature, pressure, or viscosity change in the reservoir to facilitate movement of the material through the membrane.

102. A delivery device comprising a reservoir and a membrane-penetrating apparatus, wherein the membrane-penetrating apparatus is configured to induce movement of a material through a membrane of a membrane-enclosed object in the reservoir.

103. The device of claim 102, wherein the reservoir comprises an inlet and an outlet for fluidic movement of the object into and out of the reservoir.

104. The device of claim 102, wherein the device is configured to maintain a specific temperature, pressure, viscosity of the material prior to movement through the membrane.

105. The device of claim 102, further comprising a pump configured to maintain a flow.

106. The device of claim 102, wherein the penetrating apparatus is an injector.

107. The device of claim 106, wherein the injector is configured to pierce the membrane-enclosed object to: inject material into the membrane-enclosed object, extract material from the membrane-enclosed object, or inject material into the membrane-enclosed object and extract material from the membrane-enclosed object.

108. The device of claim 102, wherein the device is a high-throughput injector.

109. The device of claim 108, wherein the high-throughput injector is capable of injecting at least 100 objects per minute, 1,000 objects per minute, 10,000 objects per minute, or 100,000 objects per minute.

110. The device of claim 102, further comprising a system configured to collect and exchange biological fluid.

111. The device of claim 110, wherein the system is an apheresis device.

112. The device of claim 110, wherein the biological fluid is selected from the group consisting of: blood and bodily fluid.

113. The device of claim 102 further comprising a detection device configured to monitor the movement of the material and obtain cellular image data.

114. The device of claim 113, wherein the detection device comprises an imaging device.

115. The device of claim 114, wherein the imaging device is a camera.

116. The device of claim 102 further comprising a computer.

117. The device of claim 116, wherein the computer is programmed to perform one or more functions selected from the group consisting of: for storing information and regulating delivery.

118. The device of claim 116, wherein the computer is an automated machine configured to follow machine-readable instructions that facilitate the transport of the objects, injection into the objects and extraction from the objects.

119. A system for automated extracorporeal injection comprising: (a) a collection system; (b) a computer including control software for motion control and image processing; (c) a control device to control motion and immobilize one or more membrane-enclosed objects in a desired position; and (d) an injection mechanism, wherein the control device and the injection mechanism are linked to the computer to facilitate the injection of material into the objects.

120. The system of claim 119 further comprising a microscope for viewing position of the injection mechanism relative to the objects.

121. The system of claim 119, wherein the collection system is an apheresis device.

122. A high-throughput system for automated injection comprising: (a) a computer including control software for motion control and image processing; (b) a control device to control motion and immobilize one or more membrane-enclosed objects in a desired position; (c) an injection mechanism; and (d) a microscope for viewing the position of the injection mechanism relative to the objects; wherein the control device, the injection mechanism and the microscope are linked to the computer to enable the injection into the objects.

123. A delivery device for delivering cells, subcellular components, fusogens, fusosomes, or fusosome compositions into a biological tissue, the delivery device comprising:
a reservoir comprising one or more selected contents from the group consisting of: cells, subcellular components, fusogens, fusosomes, and fusosome compositions; and
a material delivery unit in connection with the reservoir, the material delivery unit configured to transfer the contents from the reservoir to the tissue.

124. A method of delivering cells, subcellular components, fusogens, fusosomes, or fusosome compositions into a biological tissue, the method comprising:
positioning the delivery device according to claim 113 adjacent to, within, or partially within biological tissue; and
controlling the delivery device to transfer the cells, subcellular components, fusogens, fusosomes, or fusosome compositions from the reservoir to the biological tissue.

125. A delivery device for transfer of subcellular components, fusogens, fusosomes, or fusosome compositions across one or more membrane-enclosed objects, the delivery device comprising:
a first reservoir adapted and configured to hold unpermeabilized membrane-enclosed objects;
a permeabilizing module in communication with the first reservoir; and
a second reservoir containing subcellular components, fusogens, fusosomes, or fusosome compositions, the second reservoir in communication with the first reservoir.

126. A method of delivering subcellular components, fusogens, fusosomes, or fusosome compositions into one or more membrane-enclosed objects, the method comprising:
introducing one or more membrane-enclosed objects into the first reservoir of the delivery device according to claim 115; and
controlling the permeabilizing module to permeabilize the membrane of the one or more membrane-enclosed objects to allow movement of the subcellular components, fusogens, fusosomes, or fusosome compositions through the membrane; and
contacting the one or more membrane-enclosed objects with the subcellular components, fusogens, fusosomes, or fusosome compositions from the second reservoir.
